(19) United States
(12) Patent Application Publication
(10) Pub. No.: US 2003/0114469 A1
(43) Pub. Date: Jun. 19, 2003

(54) Combinations

(76) Inventor: David Saul Cohen, New Providence, NJ (US)

Correspondence Address: THOMAS HOXIE, NOVARTIS, PATENT AND TRADEMARK DEPARTMENT, ONE HEALTH PLAZA 430/2, EAST HANOVER, NJ, 07936-1080, US

(21) Appl. No.: 10/231,427

(22) Filed: Aug. 28, 2002

Related U.S. Application Data
(60) Provisional application No. 60/325,485, filed on Sep. 27, 2001.

(51) Int. Cl. A61K 31/522

(52) U.S. Cl. 514/263.22

(57) ABSTRACT

The present invention relates to a pharmaceutical composition, comprising

(a) a phosphodiesterase 5 inhibitor or a pharmaceutically acceptable salt thereof and

(b) at least one of the active ingredients selected from the group consisting of

(i) an anti-diabetic agent;

(ii) HMG-Co-A reductase inhibitors;

(iii) an anti-hypertensive agent; and

(iv) a serotonin reuptake inhibitor (SSRI)

or, in each case, or a pharmaceutically acceptable salt thereof; and

a pharmaceutically acceptable carrier. The pharmaceutical composition may be employed for the treatment of sexual dysfunction, hyperglycemia, hyperinsulinaemia, hyperlipidaemia, hypertriglyceridemia, diabetes, insulin resistance, impaired glucose metabolism, conditions of impaired glucose tolerance (IGT), conditions of impaired fasting plasma glucose, obesity, diabetic retinopathy, diabetic nephropathy, glomerulosclerosis, diabetic neuropathy, syndrome X, erectile dysfunction, coronary heart disease, hypertension, especially ISH, angina pectoris, myocardial infarction, stroke, vascular restenosis, endothelial dysfunction, impaired vascular compliance, congestive heart failure.

The present invention relates to a combination, especially a pharmaceutical composition, comprising

(a) a PDE 5 inhibitor or a pharmaceutically acceptable salt thereof and

(b) at least one active ingredient selected from the group consisting of

(i) an anti-diabetic agent;

(ii) HMG-Co-A reductase inhibitors;

(iii) an anti-hypertensive agent; and

(iv) a serotonin reuptake inhibitor (SSRI)

or, in each case, a pharmaceutically acceptable salt thereof; and

a pharmaceutically acceptable carrier.

Anti-diabetic agents include insulin secretion enhancers which are active ingredients that have the property to promote the secretion of insulin from pancreatic β-cells. Examples of insulin secretion enhancers are a biguanide derivative, for example, metformin or, if appropriate, a pharmaceutically acceptable salt thereof, especially the hydrochloride thereof. Other insulin secretion enhancers include sulfonylureas (SU), especially those which promote the secretion of insulin from pancreatic β-cells by transmitting signals of insulin secretion via SU receptors in the cell membrane including, but are not limited to, tolbutamide; chlorpropamide; tolazamide; acetohexamide; 4-chloro-N-[(1-pyrolidinylamino)carbonyl]-benzensulfonamide (glycopyramide); glibenclamide (glyburide); gliclazide; 1-butyl-3-metanilylurea; carbutamide; glibonuride; glipizide; gliquidone; glisoxepid; glybuthiazole; glibuzole; glyhexamide; glymidine; glypinamide; phenbutamide; and tolylcyclamide, or pharmaceutically acceptable salts thereof.

Insulin secretion enhancers furthermore include short-acting insulin secretion enhancers, such as the phenylalanine derivative nateglinide [N-(trans-4-isopropylcyclohexylcarbonyl)-D-phenylalanine] (cf. EP 196222 and EP 526171) of the formula

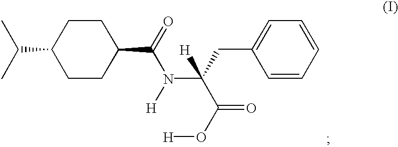

and repaglinide [(S)-2-ethoxy-4-{2-[[3-methyl-1-[2-(1-piperidinyl)phenyl]butyl]amino]-2-oxoethyl}benzoic acid]. Repaglinide is disclosed in EP 589874, EP 147850 A2, in particular Example 11 on page 61, and EP 207331 A1. It can be administered in the form as it is marketed, e.g., under the trademark NovoNorm™; calcium (2S)-2-benzyl-3-(cis-hexahydro-2-isoindolinlycarbonyl)-propionate dihydrate (mitiglinide—cf. EP 507534); furthermore representatives of the new generation of SUs such as glimepiride (cf. EP 31058); in free or pharmaceutically acceptable salt form. The term nateglinide likewise comprises crystal modifications such as disclosed in EP 0526171 B1 or U.S. Pat. No. 5,488,510, respectively, the subject matter of which, especially with respect to the identification, manufacture and characterization of crystal modifications, is herewith incorporated by reference to this application, especially the subject matter of claims 8-10 of said U.S. patent (referring to H-form crystal modification) as well as the corresponding references to the B-type crystal modification in EP 196222 B1 the subject matter of which, especially with respect to the identification, manufacture and characterization of the B-form crystal modification. Preferably, in the present invention, the B- or H-type, more preferably the H-type, is used. Nateglinide can be administered in the form as it is marketed, e.g., under the trademark STARLIX™.

Insulin secretion enhancers likewise include the long-acting insulin secretion enhancer DPP-IV inhibitors, glucagon-like peptide-1 (GLP-1) and GLP-1 agonists.

DPP-IV is responsible for inactivating GLP-1. More particularly, DPP-IV generates a GLP-1 receptor antagonist and thereby shortens the physiological response to GLP-1. GLP-1 is a major stimulator of pancreatic insulin secretion and has direct beneficial effects on glucose disposal.

The DPP-IV inhibitor can be peptidic or, preferably, non-peptidic. DPP-IV inhibitors are in each case generically and specifically disclosed, e.g., in WO 98/19998, DE 196 16 486 A1, WO 00/34241 and WO 95/15309, in each case in particular in the compound claims and the final products of the working examples, the subject-matter of the final products, the pharmaceutical preparations and the claims are hereby incorporated into the present application by reference to these publications. Preferred are those compounds that are specifically disclosed in Example 3 of WO 98/19998 and Example 1 of WO 00/34241, respectively.

GLP-1 is a insulinotropic proteine which was described, e.g., by W. E. Schmidt et al., Diabetologia, 1985, 28:704-707 and in U.S. Pat. No. 5,705,483.

The term “GLP-1 agonists” used herein means variants and analogs of GLP-1(7-36)NH₂ which are disclosed in particular in U.S. Pat. Nos. 5,120,712, 5,118,666, 5,512,549, WO 91/11457 and by C. Orskov et al., J. Biol. Chem., 1989, 264:12826. The term “GLP-1 agonists” comprises especially compounds like GLP-1(7-37), in which compound the carboxy-terminal amide functionality of Arg³⁶ is displaced with Gly at the 37^{th} position of the GLP-1(7-36)NH₂ molecule and variants and analogs thereof including GLN⁹-GLP-1(7-37), D-GLN⁹-GLP-1(7-37), acetyl LYS⁹-GLP-1(7-37), LYS¹⁸-GLP-1(7-37) and, in particular, GLP-1(7-37)OH, VAL⁸-GLP-1(7-37), GLY⁸-GLP-1(7-37), THR⁸-GLP-1(7-37), MET⁸-GLP-1(7-37) and 4-imidazopropionyl-GLP-1. Special preference is also given to the GLP agonist analog exendin-4, described by Greig et al., Diabetologia, 1999, 42:45-50.

An insulin sensitivity enhancer restores impaired insulin receptor function to reduce insulin resistance and consequently enhance the insulin sensitivity.

An appropriate insulin sensitivity enhancer is, for example, an appropriate hypoglycemic thiazolidinedione derivative (glitazone).

An appropriate glitazone is, for example, (S)-((3,4-dihydro-2-(phenyl-methyl)-2H-1-benzopyran-6-yl)methyl-thiazolidine-2,4-dione (englitazone), 5-{[4-(3-(5-methyl-2-phenyl-4-oxazolyl)-1-oxopropyl)-phenyl]-methyl}-thiazolidine-2,4-dione (darglitazone), 5-{[4-(1-methylcyclohexyl)methoxy)-phenyl]methyl}-thiazolidine-2,4-dione (ciglitazone), 5-{[4-(2-(1-indolyl)ethoxy)phenyl]methyl}-thiazolidine-2,4-dione (DRF2189), 5-{4-[2-(5-methyl-2-phenyl-4-oxazolyl)-ethoxy)]benzyl}-thiazolidine-2,4-dione (BM-13.1246), 5-(2-naphthylsulfonyl)thiazolidine-2,4-dione (AY-31637), bis{4-[(2,4-dioxo-5-thiazolidinyl)methyl]phenyl}methane (YM268), 5-{4-[2-(5-methyl-2-phenyl-4-oxazolyl)-2-hydroxyethoxy]benzyl}-thiazolidine-2,4-dione (AD-5075), 5-[4-(1-phenyl-1-cyclopropanecarbonylamino)-benzyl]-thiazolidine-2,4-dione (DN-108) 5-{[4-(2-(2,3-dihydroindol-1-yl)ethoxy)phenyl]methyl}-thiazolidine-2,4-dione, 5-[3-(4-chloro-phenyl])-2-propynyl]-5-phenylsulfonyl)thiazolidine-2,4-dione, 5-[3-(4-chlorophenyl])-2-propynyl]-5-(4-fluorophenyl-sulfonyl)thiazolidine-2,4-dione, 5-{[4-(2-(methyl-2-pyridinyl-amino)-ethoxy)phenyl]methyl}-thiazolidine-2,4-dione (rosiglitazone), 5-{[4-(2-(5-ethyl-2-pyridyl)ethoxy)phenyl]-methyl}thiazolidine-2,4-dione (pioglitazone), 5-{[4-((3,4-dihydro-6-hydroxy-2,5,7,8-tetramethyl-2H-1-benzopyran-2-yl)methoxy)-phenyl]-methyl}-thiazolidine-2,4-dione (troglitazone), 5-[6-(2-fluoro-benzyloxy)naphthalen-2-ylmethyl]-thiazolidine-2,4-dione (MCC555), 5-{[2-(2-naphthyl)-benzoxazol-5-yl]-methyl}thiazolidine-2,4-dione (T-174) and 5-(2,4-dioxothiazolidin-5-ylmethyl)-2-methoxy-N-(4-trifluoromethyl-benzyl)benzamide (KRP297). Preferred are pioglitazone, rosiglitazone and troglitazone.

Other anti-diabetic agents include, insulin signalling pathway modulators, like inhibitors of protein tyrosine phosphatases (PTPases), antidiabetic non-small molecule mimetic compounds and inhibitors of glutamine-fructose-6-phosphate amidotransferase (GFAT); compounds influencing a dysregulated hepatic glucose production, like inhibitors of glucose-6-phosphatase (G6Pase), inhibitors of fructose-1,6-bisphosphatase (F-1,6-BPase), inhibitors of glycogen phosphorylase (GP), glucagon receptor antagonists and inhibitors of phosphoenolpyruvate carboxykinase (PEPCK); pyruvate dehydrogenase kinase (PDHK) inhibitors; inhibitors of gastric emptying; insulin; inhibitors of GSK-3; retinoid X receptor (RXR) agonists; agonists of Beta-3 AR; agonists of uncoupling proteins (UCPs); non-glitazone type PPARγ agonists; dual PPARγ/PPARα agonists; antidiabetic vanadium containing compounds; incretin hormones, like GLP-1 and GLP-1 agonists; β-cell imidazoline receptor antagonists; miglitol; and α₂-adrenergic antagonists; in which the active ingredients are present in each case in free form or in the form of a pharmaceutically acceptable salt.

The term “insulin signalling pathway modulators” as defined herein relates in particular to inhibitors of PTPase, antidiabetic non-small molecule mimetic compounds and inhibitors of GFAT.

Examples of “inhibitors of PTPase” include, but are not limited to, those disclosed in U.S. Pat. Nos. 6,057,316, 6,001,867, WO 99/58518, WO 99/58522, WO 99/46268, WO 99/46267, WO 99/46244, WO 99/46237, WO 99/46236, WO 99/15529 and by Poucheret et al., Mol. Cell Biochem., 1998, 188:73-80.

The term “antidiabetic non-small molecule mimetic compounds” as defined herein means compounds as disclosed in Science, 1999, 284:974-97, especially L-783,281; and WO 99/58127, especially CLX-901.

Examples of “inhibitors of GFAT” include, but are not limited to, those disclosed in Mol. Cell. Endocrinol., 1997,135(1):67-77.

The term “compounds influencing a dysregulated hepatic glucose production” as defined herein relates in particular to inhibitors of glucose-6-phosphatase (G6Pase), inhibitors of fructose-1,6-bisphosphatase (F-1,6-BPase), inhibitors of glycogen phosphorylase (GP), glucagon receptor antagonists and inhibitors of phosphoenolpyruvate carboxykinase (PEPCK).

The term “inhibitors of G6Pase” used herein means a compound or composition which reduces or inhibits hepatic gluconeogenesis by decreasing or inhibiting the activity of G6Pase. Examples of such compounds are disclosed in WO 00/14090, WO 99/40062, WO 98/40385, EP 682024 and Diabetes, 1998, 47:1630-1636.

The term “inhibitors of F-1,6-Bpase” used herein means a compound or composition which reduces or inhibits hepatic gluconeogenesis by decreasing or inhibiting the activity of F-1,6-BPase. Examples of such compounds are disclosed in WO 00/14095, WO 99/47549, WO 98/39344, WO 98/39343 and WO 98/39342.

The term “inhibitors of GP” as used herein means a compound or composition which reduces or inhibits hepatic glycogenolysis by decreasing or inhibiting the activity of GP. Examples of such compounds are disclosed in EP 978279, U.S. Pat. No. 5,998,463, WO 99/26659, EP 846464, WO 97/31901, WO 96/39384, WO 96/39385; and in particular CP-91149 as described in Proc. Natl. Acad. Sci. USA, 1998, 95:1776-1781.

The term “glucagon receptor antagonists” as used herein relates in particular to the compounds described in WO 98/04528, especially BAY27-9955, and those described in Bioorg. Med. Chem. Lett., 1992, 2:915-918, especially CP-99711; J. Med. Chem., 1998, 41:5150-5157, especially NNC 92-1687; and J. Biol. Chem., 1999, 274:8694-8697, especially L-168,049; and compounds disclosed in U.S. Pat. No. 5,880,139, WO 99/01423, U.S. Pat. No. 5,776,954, WO 98/22109, WO 98/22108, WO 98/21957 and WO 97/16442.

The term “inhibitors of PEPCK” used herein means a compound or composition which reduces or inhibits hepatic gluconeogenesis by decreasing or inhibiting the activity of PEPCK. Examples of such compounds are disclosed in U.S. Pat. No. 6,030,837 and Mol. Biol. Diabetes, 1994, 2:283-99.

The term “PDHK inhibitors” as used herein means inhibitors of pyruvate dehydrogenase kinase and include, but are not limited to, those compounds disclosed by Aicher et al., J. Med. Chem., 1999, 42:2741-2746.

Examples of “inhibitors of gastric emptying” other than GLP-1 include, but are not limited to, those disclosed in J. Clin. Endocrinol. Metab., 2000, 85(3):1043-1048, especially CCK-8; and in Diabetes Care, 1998, 21:897-893, especially Amylin and analogs thereof, e.g., Pramlintide. Amylin is also described, e.g., by Kolterman et al., Diabetologia, 1996, 39:492-499.

Insulin is available from different providers under different tradenames, e.g., Berlinsulin© (Berlin-Chemie), Huminsulin© (Eli Lilly), Insulin Actrapid© (Novo Nordisk) or Insuman© (Aventis).

Examples of “inhibitors of GSK-3” include, but are not limited to, those disclosed in WO 00/21927 and WO 97/41854.

By “RXR agonist” is meant a compound or composition which when combined with RXR homodimers or heterodimers increases the transcriptional regulation activity of RXR, as measured by an assay known to one skilled in the art, including, but not limited to, the “co-transfection” or “cis-trans” assays described or disclosed in U.S. Pat. Nos. 4,981,784, 5,071,773, 5,298,429, 5,506,102, WO 89/05355, WO 91/06677, WO 92/05447, WO 93/11235, WO 95/18380, PCT/US93/04399, PCT/US94/03795 and CA 2,034,220, which are incorporated by reference herein. It includes, but is not limited to, compounds that preferentially activate RXR over RAR (i.e., RXR specific agonists), and compounds that activate both RXR and RAR (i.e., pan agonists). It also includes compounds that activate RXR in a certain cellular context but not others (i.e., partial agonists). Compounds disclosed or described in the following articles, patents and patent applications which have RXR agonist activity are incorporated by reference herein: U.S. Pat. Nos. 5,399,586 and 5,466,861, WO 96/05165, PCT/US95/16842, PCT/US95/16695, PCT/US93/10094, WO 94/15901, PCT/US92/11214, WO 93/11755, PCT/US93/10166, PCT/US93/10204, WO 94/15902, PCT/US93/03944, WO 93/21146, provisional applications Nos. 60,004,897 and 60,009,884, Boehm et al., J. Med. Chem., 1994, 38(16):3146-3155; Boehm et al., J. Med. Chem., 1994, 37(18):2930-2941; Antras et al., J. Biol. Chem., 1991, 266:1157-1161; Salazar-Olivo et al., Biochem. Biophys. Res. Commun., 1994, 204:157-263; and Safanova, Mol. Cell. Endocrin., 1994, 104:201-211. RXR specific agonists include, but are not limited to, LG 100268 (i.e., 2-[1-(3,5,5,8,8-pentamethyl-5,6,7,8-tetrahydro-2-naphthyl)-cyclopropyl]-pyridine-5-carboxylic acid) and LGD 1069 (i.e., 4-[(3,5,5,8,8-pentamethyl-5,6,7,8-tetrahydro-2-naphthyl)-2-carbonyl]-benzo ic acid), and analogs, derivatives and pharmaceutically acceptable salts thereof. The structures and syntheses of LG 100268 and LGD 1069 are disclosed in Boehm et al., J. Med. Chem., 1994, 38(16):3146-3155, incorporated by reference herein. Pan agonists include, but are not limited to, ALRT 1057 (i.e., 9-cis retinoic acid), and analogs, derivatives and pharmaceutically acceptable salts thereof.

Examples of “agonists of Beta-3 AR” include, but are not limited to, CL-316,243 (Lederle Laboratories) and those disclosed in WO 99/29672, WO 98/32753, WO 98/20005, WO 98/09625, WO 97/46556, WO 97/37646 and U.S. Pat. No. 5,705,515.

The term “agonists of UCPs” used herein means agonists of UCP-1, preferably UCP-2 and even more preferably UCP-3. UCPs are disclosed in Vidal-Puig et al., Biochem. Biophys. Res. Commun., 1997, 235(1):79-82. Such agonists are a compound or composition which increases the activity of UCPs.

“Non-glitazone type PPARγ agonists” are especially N-(2-benzoylphenyl)-L-tyrosine analogues, e.g., GI-262570, and JTT501.

The term “dual PPARγ/PPARα agonists” as used herein means compounds which are at the same time PPARγ and PPARα agonists. Preferred dual PPARγ/PPARα agonists are especially those ω-[(oxoquinazolinylalkoxy)phenyl]alkanoates and analogs thereof, the compound NN622 described in U.S. Pat. No. 6,054,453, example 22; very especially the compound DRF-554158, described in WO 99/08501 and the compound NC-2100 described by Fukui, Diabetes, 2000, 49(5):759-767.

Preferably, the “antidiabetic vanadium containing compound” is a physiologically tolerable vanadium complex of a bidentate monoprotic chelant, wherein said chelant is an α-hydroxypyrone or α-hydroxypyridinone, especially those disclosed in the Examples of U.S. Pat. No. 5,866,563, of which the working examples are hereby incorporated by reference, or a pharmaceutically acceptable salt thereof.

The term “incretin hormones” as used herein relates in particular to GLP-1 or GLP-1 agonists. GLP-1 is a insulinotropic proteine which was described, e.g., by Schmidt et al., Diabetologia, 1985, 28:704-707 and in U.S. Pat. No. 5,705,483. The term “GLP-1 agonists” used herein means variants and analogs of GLP-1(7-36)NH₂ which are disclosed in particular in U.S. Pat. Nos. 5,120,712, 5,118,666, 5,512,549, WO 91/11457 and by Orskov et al., J. Biol. Chem., 1989, 264:12826. The term “GLP-1 agonists” comprises especially compounds like GLP-1(7-37), in which compound the carboxy-terminal amide functionality of Arg³⁶ is displaced with Gly at the 37^{th} position of the GLP-1(7-36)NH₂ molecule and variants and analogs thereof including GLN⁹-GLP-1(7-37), D-GLN⁹-GLP-1(7-37), acetyl LYS⁹-GLP-1(7-37), LYS¹⁸-GLP-1(7-37) and, in particular, GLP-1(7-37)OH, VAL⁸-GLP-1(7-37), GLY⁸-GLP-1(7-37), THR⁸-GLP-1(7-37), MET⁸-GLP-1(7-37) and 4-imidazopropionyl-GLP-1. Special preference is also given to the GLP agonist analog exendin-4, described by Greig et al., Diabetologia, 1999, 42:45-50.

The term “β-cell imidazoline receptor antagonists” as used herein means compounds as those described in WO 00/78726 and by Wang et al., J. Pharmacol. Exp. Ther., 1996, 278:82-89, e.g., PMS 812.

Miglitol is (2R, 3R, 4R, 5S)-1-(2-hydroxyethyl)-2-(hydroxymethyl)-3,4,5-piperidinetriol and is described in U.S. Pat. No. 4,639,436. The 1-deoxynojirimycin derivative miglitol can be administered in the form as it is marketed, e.g., under the trademark DIASTABOL 50™.

Examples of “α₂-adrenergic antagonists” include, but are not limited to, midaglizole described in Diabetes, 1987, 36:216-220.

The insulin signalling pathway modulators, compounds influencing a dysregulated hepatic glucose production, pyruvate dehydrogenase kinase (PDHK) inhibitors, inhibitors of gastric emptying, inhibitors of GSK-3, RXR agonists, agonists of Beta-3 AR, agonists of UCPs, non-glitazone type PPARγ agonists, dual PPARγ/PPARα agonists, antidiabetic vanadium containing compounds, incretin hormones, β-cell imidazoline receptor antagonists, miglitol, and α₂-adrenergic antagonists are in each case generically and specifically disclosed in the documents cited above, in each case in particular in the compound claims and the final products of the working examples, the subject-matter of the final products, the pharmaceutical preparations and the claims are hereby incorporated into the present application by reference to these publications. Comprised are likewise the corresponding stereoisomers as well as the corresponding crystal modifications, e.g., solvates and polymorphs, which are disclosed therein and, where applicable, all pharmaceutically acceptable salts thereof.

Any person skilled in the art is fully enabled to identify the active agents and, based on these references, likewise enabled to manufacture and test the pharmaceutical indications and properties in standard test models, both in vitro and in vivo.

HMG-Co-A reductase inhibitors (also called β-hydroxy-β-methylglutaryl-co-enzyme-A reductase inhibitors) are understood to be those active agents that may be used to lower the lipid levels including cholesterol, especially LDL-cholesterol, in blood.

The class of HMG-Co-A reductase inhibitors comprises compounds having differing structural features. For example, mention may be made of the compounds that are selected from the group consisting of atorvastatin (cf. EP 680320), cerivastatin (cf. EP 491226), fluvastatin (cf. U.S. Pat. No. 5,354,772), pitavastatin (cf. EP 304063), lovastatin (cf. EP 22478), pravastatin (cf. UK 2077264), rosuvastatin (S 4522) (Wantanabe, Bioorg. Med. Chem., 1997, 5(2):437-444) and simvastatin (cf. EP 33538), or, in each case, a pharmaceutically acceptable salt thereof.

Preferred HMG-Co-A reductase inhibitors are those agents that have been marketed, most preferred is fluvastatin, atorvastatin, pravastatin or simvastatin or, in each case, a pharmaceutically acceptable salt thereof.

Anti-hypertensive agents include angiotensin converting enzyme inhibitors (ACE-inhibitors) and AT₁ receptor antagonists. The interruption of the enzymatic degradation of angiotensin I to angiotensin II with ACE-inhibitors is a successful variant for the regulation of blood pressure and thus also makes available a therapeutic method for the treatment of congestive heart failure.

The class of ACE inhibitors comprises compounds having differing structural features. For example, mention may be made of the compounds which are selected from the group consisting alacepril (cf. EP 7477), benazepril (cf. EP 72352), benazeprilat (cf. EP 72352), captopril (cf. U.S. Pat. No. 4,105,776), ceronapril (cf. EP 229520), cilazapril (cf. EP 94095), delapril (cf. EP 51391), enalapril (cf. EP 12401), enaprilat (cf. EP 12401), fosinopril (cf. EP 53902), imidapril (cf. EP 95163), lisinopril (cf. EP 12401), moveltipril (cf. ZA 82/3779), perindopril (cf. EP 49658), quinapril (cf. EP 49605), ramipril (cf. EP 79022), spirapril (cf. EP 50800), temocapril (cf. EP 161801), and trandolapril (cf. EP 551927), or, in each case, a pharmaceutically acceptable salt thereof.

Preferred ACE inhibitors are those agents that have been marketed, most preferred are benazepril and enalapril.

The corresponding active ingredients or a pharmaceutically acceptable salts thereof may also be used in form of a solvate, such as a hydrate or including other solvents, used for crystallization.

The compounds to be combined can be present as pharmaceutically acceptable salts. If these compounds have, for example, at least one basic center, they can form acid addition salts. Corresponding acid addition salts can also be formed having, if desired, an additionally present basic center. The compounds having an acid group (for example, COOH) can also form salts with bases.

The class of AT₁ receptor antagonists comprises compounds having differing structural features, essentially preferred are the non-peptidic ones. For example, mention may be made of the compounds which are selected from the group consisting of valsartan, losartan, candesartan, eprosartan, irbesartan, saprisartan, tasosartan, telmisartan, the compound with the designation E-1477 of the following formula

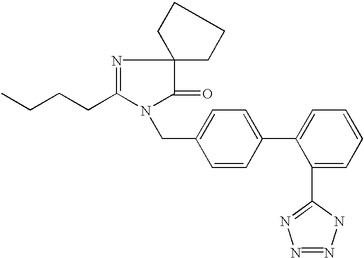

the compound with the designation SC-52458 of the following formula

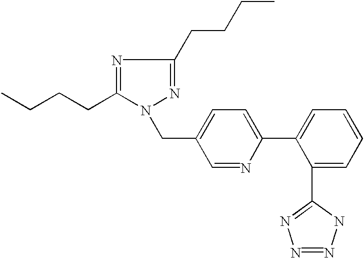

and the compound with the designation the compound ZD-8731 of the following formula

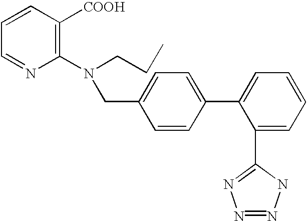

or, in each case, a pharmaceutically acceptable salt thereof.

Preferred AT₁-receptor antagonist are those agents which have been marketed, most preferred is valsartan or a pharmaceutically acceptable salt thereof.

Additional anti-hypertensive agents include adrenergic blockers, diuretics, neutral endo-peptidases inhibitors, endothelin converting enzymes inhibitors, endothelin receptor antagonists, adrenergic stimulants, alpha/beta adrenergic blockers beta adrenergic blocking agents, calcium channel blockers, diuretics, rauwolfia derivatives and vasodilators or any combination thereof.

Serotonin reuptake inhibitors (SSRIs), include, for example, fluvoxamine; fluoxetine; paroxetine; sertraline; citalopram; venlafaxine; cericlamine; duloxetine; milnacipran; nefazodone; and cyanodothiepin (See The Year Drugs News, 1995 Edition, pp. 47-48 by Prous J. R.) and WO 97/29739.

The structure of the active agents identified by generic or tradenames may be taken from the actual edition of the standard compendium “The Merck Index” or from databases, e.g., Patents International (e.g., IMS World Publications). The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

PDE5 inhibitors include compounds of formula

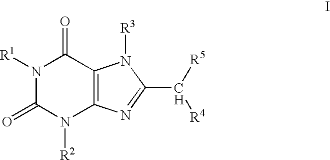

in free or salt form, where

R¹ is hydrogen or alkyl optionally substituted by hydroxy, alkoxy, or alkylthio;

R² is hydrogen, alkyl, hydroxyalkyl, alkylcarbonyloxyalkyl, alkoxyalkyl, alkylthioalkyl, alkenyl, cycloalkylalkyl, heterocyclylalkyl, aralkyl in which the aryl ring thereof is optionally fused to a 5-membered heterocyclic group or is optionally substituted by one or more substituents selected from alkoxy, amino, alkylamino, dialkylamino, acylamino, halogen, hydroxy, aminosulfonyl, alkylaminosulfonyl, dialkylaminosulfonyl, alkylsulfonylamino or dialkylaminosulfonylamino;

R³ is hydrogen or alkyl optionally substituted by hydroxy, alkoxy, or alkylthio;

R⁴ is hydrogen or alkyl;

R⁵ is a quinolinyl, isoquinolinyl or oxodihydroisoquinolinyl group optionally fused to a 5-membered heterocyclic group and optionally substituted by one or more substituents selected from halogen, cyano, hydroxy, alkyl, hydroxyalkyl, alkoxyalkyl, alkylthioalkyl, alkoxy, alkylthio, alkenyl, alkoxycarbonyl, alkynyl, carboxyl, acyl, a group of formula —N(R⁶)R⁷, aryl optionally substituted by one or more substituents selected from halogen or alkoxy, or heteroaryl having 5 or 6 ring atoms, attached through a ring carbon atom to the indicated carbon atom; and

R⁶ and R⁷ are each independently hydrogen or alkyl optionally substituted by hydroxy or alkoxy or one of R⁶ and R⁷ is hydrogen and the other is acyl, or R⁶ and R⁷ together with the nitrogen atom to which they are attached denote a 5- or 6-membered heterocyclyl group.

“Alkyl” as used herein denotes straight chain or branched alkyl, which may be, for example, C₁-C₁₀-alkyl such as methyl, ethyl, n-propyl, isopropyl, n-butyl, isobutyl, sec-butyl, tert-butyl, straight or branched pentyl, straight or branched hexyl, straight or branched heptyl, straight or branched octyl, straight or branched nonyl or straight or branched decyl. Preferably alkyl is C₁-C₈-alkyl.

“Alkoxy” as used herein denotes straight chain or branched alkoxy which may be, for example, C₁-C₁₀-alkoxy such as methoxy, ethoxy, n-propoxy, isopropoxy, n-butoxy, isobutoxy, sec-butoxy, tert-butoxy, straight or branched pentoxy, straight or branched hexyloxy, straight or branched heptyloxy, straight or branched octyloxy, straight or branched nonyloxy or straight or branched decyloxy. Preferably, alkoxy is C₁-C₄-alkoxy.

“Alkylthio” as used herein may be C₁-C₁₀-alkylthio such as methylthio, ethylthio, n-propylthio, isopropylthio, n-butylthio, sec-butylthio, isobutylthio, tert-butylthio, pentylthio, hexylthio, heptylthio, octylthio, nonylthio or decylthio. Preferably alkylthio is C₁-C₄-alkylthio.

“Alkenyl” as used herein means straight chain or branched alkenyl, which may be, for example, C₂-C₁₀-alkenyl such as vinyl, 1-propenyl, 2-propenyl, 1-butenyl, isobutenyl, or straight or branched pentenyl, hexenyl, heptenyl, octenyl, nonenyl or decenyl. Preferred alkenyl is C₂-C₄-alkenyl.

“Cycloalkylalkyl” as used herein denotes alkyl, for example, C₁-C₁₀-alkyl such as one of the C₁-C₁₀-alkyl groups hereinbefore mentioned, substituted by a C₃-C₈-cycloalkyl group such as cyclopropyl, methylcyclopropyl, cyclobutyl, cyclopentyl, cyclohexyl, methylcyclohexyl, dimethylcyclohexyl, cycloheptyl or cyclooctyl. Preferably, cycloalkylalkyl is C₃-C₆-cycloalkyl-C₁-C₄-alkyl.

“Heterocyclylalkyl” as used herein denotes alkyl, for example, C₁-C₁₀-alkyl such as one of the C₁-C₁₀-alkyl groups hereinbefore mentioned, substituted by a 5- or 6-membered heterocyclyl group having one or two hetero atoms selected from nitrogen, oxygen and sulfur in the ring, such as pyrrolyl, pyrrolidinyl, furyl, thienyl, pyridyl, piperidyl, imidazolyl, imidazolidinyl, pyrazolidinyl, piperazinyl, morpholinyl, oxazolyl, or furazanyl. Preferably, heterocyclylalkyl is C₁-C₄-alkyl substituted by a 5- or 6-membered heterocyclyl group having one or two nitrogen or oxygen atoms or one nitrogen atom and one oxygen atom in the ring.

“Aralkyl” as used herein means C₆-C₁₀-aryl-C₁-C₁₀-alkyl and may be, for example, one of the C₁-C₁₀-alkyl groups mentioned hereinbefore, particularly one of the C₁-C₄-alkyl groups, substituted by phenyl, tolyl, xylyl or naphthyl. Preferably, aralkyl is phenyl-C₁-C₄-alkyl, particularly benzyl or 2-phenylethyl.

“Acyl” as used herein denotes alkylcarbonyl, for example, C₁-C₁₀-alkylcarbonyl where C₁-C₁₀-alkyl may be one of the C₁-C₁₀-alkyl groups hereinbefore mentioned, optionally substituted by one or more halogen atoms; cycloalkylcarbonyl, for example, C₃-C₈-cycloalkylcarbonyl where C₃-C₈-cycloalkyl may be, for example, cyclopropyl, cyclobutyl, cyclopentyl, cyclohexyl, cycloheptyl or cyclooctyl; 5- or 6-membered heterocyclylcarbonyl having one or two hetero atoms selected from nitrogen, oxygen and sulfur in the ring, such as furylcarbonyl or pyridylcarbonyl; arylcarbonyl, for example, C₆-C₁₀-arylcarbonyl, such as benzoyl; or aralkylcarbonyl, for example, C₆-C₁₀-aryl-C₁-C₄-alkylcarbonyl, such as benzylcarbonyl or phenylethylcarbonyl. Preferably acyl is C₁-C₄-alkylcarbonyl.

“Alkynyl” as used herein denotes straight or branched alkynyl, for example, C₂-C₆-alkynyl, such as ethynyl, propargyl, 2-butynyl, pentynyl or hexynyl. Preferably alkynyl is C₂-C₄-alkynyl.

“Aryl” as used herein denotes a monovalent carbocylic aromatic group, for example, C₆-C₁₀-aryl, such as phenyl, phenyl substituted by one or more, e.g., one, two or three, C₁-C₄-alkyl groups, or naphthyl. Preferably aryl is phenyl.

“Heteroaryl having 5 or 6 ring atoms” as used herein denotes a monovalent aromatic heterocyclic group having 5 or 6 ring atoms of which one, two or three are selected from nitrogen, oxygen and sulfur, such as pyrrolyl, furyl, thienyl, pyridyl, pyrazolyl, imidazolyl, triazolyl, oxazolyl, isoxazolyl, thiazolyl, isothiazolyl, dithiazolyl, trithiazolyl, furazanyl, pyrazinyl, pyrimidinyl or triazinyl.

In alkylamino, dialkylamino, acylamino, dialkylaminosulfonylamino, alkylcarbonyl, alkylcarbonyloxy, alkoxycarbonyl, hydroxyalkyl, alkylthioalkyl and alkoxyalkyl, the alkyl, acyl or alkoxy groups as appropriate have the meanings hereinbefore described.

“Halogen” as used herein may be fluorine, chlorine, bromine or iodine; preferably it is fluorine, chlorine or bromine.

The 5-membered heterocyclic ring to which R⁵ as a quinolinyl, isoquinolinyl or oxodihydroisoquinolinyl group is optionally fused may be, for example, a 5-membered heterocyclic ring having one or two hetero atoms in the ring, said hetero atoms being selected from oxygen, nitrogen and sulfur. Examples of such heterocyclic rings include pyrrole, pyrroline, pyrrolidine, furan, dihydrofuran, tetrahydrofuran, thiophene, dihydrothiophene, tetrahydrothiophene, imidazole, imidazoline, imidazolidine, pyrazole, pyrazoline, pyrazolidine, dioxolane, oxazole, isoxazole, thiazole and isothiazole rings. Preferably the 5-membered heterocyclic ring is a saturated ring having two hetero atoms, preferably two oxygen or two nitrogen atoms, especially two oxygen atoms.

R⁵ as a quinolinyl group may be a 2-quinolinyl, 3-quinolinyl, 4-quinolinyl, 5-quinolinyl, 6-quinolinyl, 7-quinolinyl or 8-quinolinyl group, preferably a 4-quinolinyl, 5-quinolinyl or 8-quinolinyl group. R⁵ as an isoquinolinyl group may be a 1-isoquinolinyl, 3-isoquinolinyl, 4-isoquinolinyl, 5-isoquinolinyl, 6-isoquinolinyl, 7-isoquinolinyl, or 8-isoquinolinyl group, preferably a 1-isoquinolinyl or 4-isoquinolinyl group. In most of the especially preferred embodiments of the invention, R⁵ is a 4-isoquinolinyl group.

R⁵ as a substituted quinolinyl or isoquinolinyl group is preferably substituted by one, two, three or four of the abovementioned substituents, especially one, two or three of those substituents. The preferred substituted 4-isoquinolinyl group is preferably substituted in the 1- and/or 6- and/or 7- and/or 8-position of the isoquinoline ring system.

In especially preferred embodiments of the invention, R⁵ is a quinolinyl group of formula

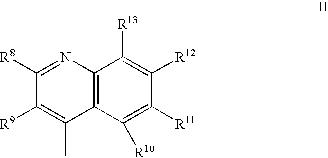

or an isoquinolinyl group of formula

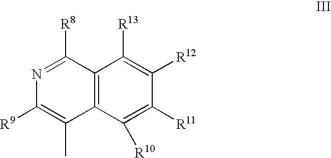

where R⁸, R⁹, R¹⁰, R¹¹, R¹² and R¹³ are each independently hydrogen or a substituent selected from halogen, cyano, hydroxy, alkyl, hydroxyalkyl, alkoxyalkyl, alkylthioalkyl, alkoxy, alkylthio, alkenyl, alkoxycarbonyl, alkynyl, carboxyl, acyl, a group of formula —N(R⁶)R⁷, aryl optionally substituted by one or more substituents selected from halogen or alkoxy, or heteroaryl having 5 or 6 ring atoms, or R¹¹ and R¹² together with the carbon atoms to which they are attached denote a 5-membered heterocyclic group having two oxygen or nitrogen atoms in the ring, and R⁶ and R⁷ are as hereinbefore defined.

R⁵ as an oxodihydroisoquinolinyl group preferably has the oxo group ortho to the ring nitrogen atom, preferably in the 1-position in the isoquinoline ring system. It is preferably linked to the remainder of the molecule of formula I via the ring carbon atom meta to the ring nitrogen atom, i.e., the 4-position in the isoquinoline ring system. An especially preferred oxodihydroisoquinolinyl group is of formula

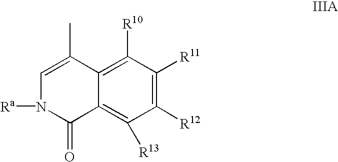

where R¹⁰, R¹¹, R¹² and R¹³ are as hereinbefore defined and R^{a} is hydrogen or C₁-C₄-alkyl.

Preferred among the compounds of formula I in free or salt form are those where

R¹ is hydrogen or C₁-C₄-alkyl optionally substituted by hydroxy, C₁-C₄-alkoxy or C₁-C₄-alkylthio;

R² is hydrogen, C₁-C₈-alkyl, hydroxy-C₁-C₈-alkyl, C₁-C₄-alkylcarbonyloxy —C₁-C₈-alkyl, C₁-C₄-alkoxy-C₁-C₈-alkyl, C₁-C₄-alkylthio-C₁-C₈-alkyl, C₂-C₄-alkenyl, C₃-C₈-cycloalkyl-C₁-C₄-alkyl, heterocyclyl-C₁-C₄-alkyl where the heterocyclyl group is a 5- or 6-membered heterocyclyl group having one or two hetero atoms selected from nitrogen and oxygen atoms in the ring, phenyl-C₁-C₄-alkyl in which the phenyl ring is optionally substituted by one or more substituents selected from C₁-C₄-alkoxy, amino, C₁-C₄-alkylamino, di(C₁-C₄-alkyl)amino, C₁-C₄-alkylcarbonylamino, halogen, C₁-C₄-alkylsulfonylamino, or di(C₁-C₄-alkyl)aminosulfonylamino, and is optionally fused to a 5-membered heterocyclic ring having two oxygen or two nitrogen atoms in the ring;

R³ is hydrogen or C₁-C₄-alkyl optionally substituted by hydroxy, C₁-C₄-alkoxy or C₁-C₄-alkylthio;

R⁴ is hydrogen or C₁-C₄-alkyl;

R⁵ is a quinolinyl, isoquinolinyl or oxodihydroisoquinolinyl group optionally fused to a 5-membered heterocyclic group having two oxygen or two nitrogen atoms in the ring and optionally substituted by one or more substituents selected from halogen, cyano, carboxy hydroxy, C₁-C₄-alkyl, hydroxy-C₁-C₄-alkyl, C₁-C₄-alkoxy-C₁-C₄-alkyl, C₁-C₄-alkylthio-C₁-C₄-alkyl, C₁-C₄-alkoxy, C₁-C₄-alkylthio, C₂-C₄-alkenyl, C₂-C₄-alkynyl, C₁-C₄-alkylcarbonyl, a group —N(R⁶)R⁷ or phenyl optionally substituted by one or more substituents selected from halogen or C₁-C₄-alkoxy; and

R⁶ and R⁷ are each independently hydrogen or C₁-C₄-alkyl optionally substituted by hydroxy or alkoxy, or one of R⁶ and R⁷ is hydrogen and the other is C₁-C₄-alkylcarbonyl, or R⁶ and R⁷ together with the nitrogen atom to which they are attached denote a 5- or 6-membered heterocyclyl group having one or two nitrogen atoms and, optionally, an oxygen atom in the ring.

Further preferred among the compounds of formula I are those where

R¹ is hydrogen or C₁-C₄-alkyl, R² is hydrogen, C₁-C₈-alkyl, hydroxy-C₁-C₈-alkyl, or C₁-C₄-alkylcarbonyloxy-C₁-C₈-alkyl, C₂-C₄-alkenyl, C₃-C₆-cycloalkyl-C₁-C₄-alkyl, heterocyclyl-C₁-C₄-alkyl where the heterocyclyl group is a 5-membered heterocyclyl group having one nitrogen or oxygen atom in the ring, phenyl-C₁-C₄-alkyl in which the phenyl ring is optionally substituted by one or two substituents selected from C₁-C₄-alkoxy, amino, C₁-C₄-alkylcarbonylamino, chlorine, bromine, C₁-C₄-alkylsulfonylamino, or di(C₁-C₄-alkyl)aminosulfonylamino and is optionally fused to a 5-membered heterocyclic ring having two oxygen atoms in the ring;

R³ is hydrogen or C₁-C₄-alkyl;

R⁴ is hydrogen or C₁-C₄-alkyl;

R⁵ is a quinolinyl group of formula II, an isoquinolinyl group of formula III or an oxodihydroisoquinolinyl group of formula IIIA, where R⁸, R⁹, R¹⁰, R¹¹, R¹² and R¹³ are each independently selected from hydrogen, halogen, cyano, carboxy, hydroxy, C₁-C₄-alkyl, hydroxy-C₁-C₄-alkyl, C₁-C₄-alkoxy-C₁-C₄-alkyl, C₁-C₄-alkylthioC₁-C₄-alkyl, C₁-C₄-alkoxy, C₁-C₄-alkylthio, C₂-C₄-alkenyl, C₂-C₄-alkynyl, C₁-C₄-alkylcarbonyl, a group —N(R⁶)R⁷ or phenyl optionally substituted by one or two substituents selected from halogen or C₁-C₄-alkoxy, or R¹¹ and R¹² together with the carbon atoms to which they are attached denote a 5-membered heterocyclic group having two oxygen atoms in the ring; and

R⁶ and R⁷ are each independently hydrogen or C₁-C₄-alkyl optionally substituted by hydroxy or alkoxy or one of R⁶ and R⁷ is hydrogen and the other is C₁-C₄-alkylcarbonyl, or R⁶ and R⁷ together with the nitrogen atom to which they are attached denote a 6-membered heterocyclyl group having one or two nitrogen atoms, or one nitrogen atom and one oxygen atom, in the ring.

Amongst the further preferred compounds hereinbefore described, especially preferred compounds are usually those in which R⁵ is an isoquinolinyl group of formula III in which R⁸ is hydrogen, C₁-C₄-alkyl, halogen, cyano, —N(R⁶)R⁷ where R⁶ and R⁷ are independently C₁-C₄-alkyl or R⁶ and R⁷ together with the nitrogen atom to which they are attached denote a 6-membered heterocyclyl group having one or two nitrogen atoms, or one nitrogen atom and one oxygen atom, in the ring, or phenyl substituted by one or two C₁-C₄-alkoxy groups; R⁹ and R¹⁰ are each independently hydrogen, C₁-C₄-alkyl or halogen; R¹¹ and R¹² are each independently hydrogen, halogen, cyano, carboxy, hydroxy, C₁-C₄-alkyl, C₁-C₄-alkoxy or C₂-C₄-alkynyl, or R¹¹ and R¹² together with the carbon atoms to which they are attached denote a 5-membered heterocycle having two oxygen atoms in the ring; and R¹³ is hydrogen or halogen.

Specific especially preferred compounds of formula I are those hereinafter described in the Examples. More preferred amongst these compounds are those of Examples 7, 10, 15, 35, 45, 49, 55, 60, 68 and 70.

Compounds of formula I may be in the form of salts, particularly pharmaceutically acceptable salts. Pharmaceutically acceptable acid addition salts of compounds of formula I include those of inorganic acids, for example, hydrohalic acids, such as hydrofluoric acid, hydrochloric acid, hydrobromic acid or hydroiodic acid, nitric acid, sulfuric acid, phosphoric acid; and organic acids, for example, aliphatic monocarboxylic acids, such as formic acid, acetic acid, trifluoroacetic acid, propionic acid and butyric acid, aliphatic hydroxy acids, such as lactic acid, citric acid, tartaric acid or malic acid, dicarboxylic acids, such as maleic acid or succinic acid, aromatic carboxylic acids, such as benzoic acid, p-chlorobenzoic acid, diphenylacetic acid or triphenylacetic acid, aromatic hydroxy acids, such as o-hydroxybenzoic acid, p-hydroxybenzoic acid, 1-hydroxynaphthalene-2-carboxylic acid or 3-hydroxynaphthalene-2-carboxylic acid, and sulfonic acids, such as methanesulfonic acid or benzenesulfonic acid. Pharmaceutically acceptable base salts of compounds of formula I where R³ is hydrogen include metal salts, particularly alkali metal or alkaline earth metal salts, such as sodium, potassium, magnesium or calcium salts, and salts with ammonia or pharmaceutically acceptable organic amines or heterocylic bases, such as ethanolamines, benzylamines or pyridine. These salts may be prepared from free compounds of formula I or other salts of compounds of formula I by known salt-forming procedures.

The present invention also provides a process for the preparation of compounds of formula I in free or salt form which comprises

1) (a) dehydrating a compound of formula

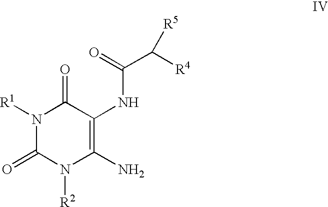

where R¹, R², R⁴ and R⁵ are as hereinbefore defined; or

(b) for the preparation of a compound of formula I in free or salt form where R³ is alkyl optionally substituted by hydroxy, alkoxy or alkylthio, reacting a compound of formula I in free or salt form with an appropriate alkylating agent; or

(c) for the preparation of a compound of formula I in free or salt form where R² is aralkyl substituted in the aryl ring by alkylsulfonylamino or dialkylaminosulfonylamino, reacting a compound of formula I in salt form where R² is aralkyl substituted by amino with, respectively, an alkylsulfonyl halide or dialkylaminosulfonyl halide; or

(d) for the preparation of a compound of formula I in free or salt form where R² is hydroxy-substituted alkyl, hydration of a compound of formula I where R² is alkenyl; or

(e) for the preparation of a compound of formula I in free or salt form where R² is alkyl substituted by alkylcarbonyloxy, appropriate esterification of a compound of formula I where R² is hydroxy-substituted alkyl; or

(f) for the preparation of a compound of formula I in free or salt form where R² is aralkyl substituted in the aryl ring by amino, hydrolysing a compound of formula I where R² is aralkyl substituted in the aryl ring by acylamino; or

(g) for the preparation of a compound of formula I in free or salt form where R⁵ is quinolinyl or isoquinolinyl substituted by hydroxy, dealkylation of a compound of formula I where R⁵ is respectively quinolinyl or isoquinolinyl substituted by alkoxy, particularly methoxy; or

(h) for the preparation of a compound of formula I in free or salt form where R⁵ is quinolinyl or isoquinolinyl substituted by halogen, halogenation of a compound of formula I where R⁵ is respectively quinolinyl or isoquinolinyl having an unsubstituted ring carbon atom available for halogenation; or

(i) for the preparation of a compound of formula I in free or salt form where R² is a cyclopropyl group, optionally substituted by alkyl, subjecting a compound of formula I where R² is alkenyl to a Simmons Smith cyclopropanation reaction; and

2) recovering the resulting product of formula I in free or salt form.

Process (a) may be carried out by heating, or by reaction with an inorganic or organic base. It may be effected in an organic or aqueous solvent or mixed aqueous/organic solvent. The reaction with base may be carried out at ambient temperature or, more conveniently, at elevated temperature. The reaction is preferably carried out by treatment with aqueous alkali metal hydroxide in an alcoholic solvent at elevated temperature, for example as described hereinafter in the Examples. The compound of formula IV is preferably a compound where R⁵ is a group of formula II or III. Compounds of formula IV may be prepared by reacting a compound of formula

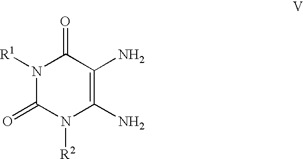

where R¹ and R² are as hereinbefore defined, with a compound of formula

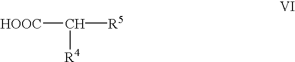

or an amide-forming derivative thereof, where R⁴ and R⁵ are as hereinbefore defined. The reaction may be effected by treating the carboxylic acid of formula VI with a peptide coupling agent to form in situ an activated ester or mixed anhydride, followed by treatment with the compound of formula V in an organic, e.g., dipolar aprotic, or mixed aqueous-organic (e.g., chlorohydrocarbon) solvent. The latter treatment may be carried out at sub-ambient, ambient or elevated temperature, conveniently at ambient temperature. Preferably, the acid of formula VI is treated with a carbodiimide derivative in the presence of hydroxybenzotriazole and, optionally, a base, or is treated with a benzotriazolyl-(trisdialkylamino)-oxyphosphonium salt. The resulting intermediate is preferably treated with the compound of formula V in a dipolar aprotic solvent or mixed chlorohydrocarbon-aqueous solvent at ambient temperature. Procedures may be as hereinafter described in the Examples.

Compounds of formula V may be prepared by reduction of a compound of formula

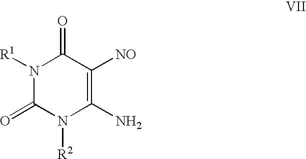

where R¹ and R²are as hereinbefore defined.

The reduction may be effected using known procedures, for example by treating the compound of formula VII with a reducing agent in an organic or aqueous solvent. The reaction may be carried out at ambient or, more conveniently, at elevated temperature. Preferred reducing agents are alkali metal dithionite salts in aqueous media or hydrogen in the presence of a noble metal catalyst. Treatment with sodium dithionite in aqueous solution at 80-90° C. is particularly preferred.

Compounds of formula VII may be prepared by nitrosation of a compound of formula

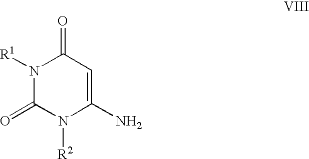

where R¹and R² are as hereinbefore defined, for example, with an organic or inorganic nitrosating agent in an organic or aqueous or mixed organic-aqueous solvent. Nitrosation may be effected using known procedures at sub-ambient, ambient or elevated temperature, preferably with an alkali metal nitrite, such as sodium nitrite in the presence of an acid such acetic acid at sub-ambient or ambient temperature, preferably in a mixed alcoholic-aqueous solvent, such as aqueous ethanol.

Compounds of formula VIII may be prepared by reacting a compound of formula

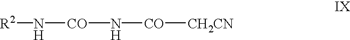

where R² is as hereinbefore defined with an inorganic or organic base to effect cyclisation, followed, where R¹ is an optionally substituted alkyl group, by reaction with an alkylating agent. The cyclisation reaction may be effected using conventional procedures. It is conveniently carried out in an aqueous, organic or mixed organic-aqueous solvent. Reaction may be effected at ambient or, more conveniently, elevated temperature. The base is preferably an alkali metal hydroxide, especially sodium hydroxide, which is preferably reacted in a mixed aqueous-alcoholic solvent, preferably at elevated temperature, e.g., 80-90° C. The optional alkylation step can be effected using known procedures, for example in the presence of an inorganic or organic base, for example, in an aqueous, organic or mixed aqueous-organic solvent. Alkylation may be carried out at sub-ambient temperature or, more conveniently, at ambient or elevated temperature. Preferred alkylating agents are alkyl iodides or, especially, dialkyl sulfates. Preferred bases are alkali metal hydroxides in aqueous alcoholic solvents, especially aqueous ethanol.

Compounds of formula IX may be prepared by reacting a compound of formula

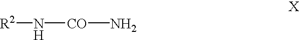

with cyanoacetic acid or an amide-forming derivative thereof, such as an ester or acid halide thereof, preferably the acid or its ethyl ester. The reaction may be effected using known procedures, for example, in an organic solvent, preferably an anhydride such as acetic anhydride. The reaction temperature may be ambient or, more conveniently, elevated temperature, e.g., 65-70° C.

Compounds of formula X may be prepared using conventional procedures, for example, from an isocyanate R²NCO by reaction with gaseous or aqueous ammonia or from an amine R²NH₂ by reaction with a metal cyanate, for example as hereinafter described in the Examples.

Compounds of formula VIII where R¹ is alkyl optionally substituted by hydroxy, alkoxy or alkylthio and R² is as hereinbefore defined other than hydrogen, may be prepared by hydrogenolysis of a compound of formula

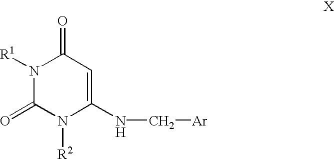

where R¹ is alkyl optionally substituted by hydroxy, alkoxy or alkylthio, R² is as hereinbefore defined other than hydrogen and Ar is phenyl optionally substituted by one or more C₁-C₄-alkoxy, preferably methoxy, groups. The hydrogenolysis may be carried out in a known manner, e.g., by treatment with hydrogen or a source of hydrogen and a metal catalyst, such as a platinum or, preferably, palladium catalyst. The reaction may be carried out in an organic solvent. The reaction temperature may be ambient or elevated. Preferably hydrogenolysis is effected using palladium black in formic acid, e.g., as hereinafter described in the Examples.

Compounds of formula XI may be prepared by reacting a compound of formula

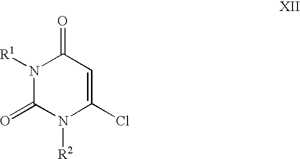

where R¹and R² are as hereinbefore defined for formula XI, with a compound of formula ArCH₂NH₂ where Ar is as hereinbefore defined. The reaction may be carried out in a known manner, e.g., in an organic solvent, preferably an alcohol, such as n-butanol, at ambient or elevated temperature, or analogously as hereinafter described in the Examples.

Compounds of formula XII may be prepared by reacting a compound of formula

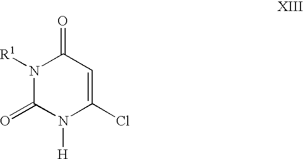

where R¹ is as hereinbefore defined for formula XI, with a compound of formula R²X where R² is as hereinbefore defined for formulae XI and X is halogen or hydroxy, where X is hydroxy, the reaction being carried out in the presence of activating reagents, preferably an azodicarboxylate, such as di-t-butyl azodicarboxylate together with a triarylphosphine, such as diphenylpyridylphosphine. The reaction may be carried out in an organic solvent, preferably an ether, such as dioxan. The reaction temperature may be sub-ambient or, preferably, ambient or elevated temperature. The reaction may be carried out using the procedure of Mitsonobu, Synthesis, 1981:1; or analogously as hereinafter described in the Examples. Compounds of formula XIII are known or may be prepared by known procedures.

Compounds of formula VI may be prepared, for example, (i) from benzaldehyde or a substituted benzadehyde using the procedure of Dyke et al., Tetrahedron, 1968, 24:1467 or (ii) from an optionally substituted, N-protected 1,2-dihydroisoquinoline by reaction with a 2-oxo-carboxylic acid using the procedure of Dyke et al., Tetrahedron, 1968, 24:1467, optionally followed by conversion of the resulting carboxylic acid into a methyl ester and then an alkali metal salt using the procedure of March, Advanced Organic Chemistry, 4^{th} Edition, Wiley, NY, 1992:393 and 378 or (iii) from an optionally substituted quinoline or isoquinoline by reaction with a hydride reducing agent followed by a 2-oxo-carboxylic ester using the procedure of Minter et al., J. Org. Chem., 1988, 53:2653 or (iv) by introducing substituents onto the N-containing ring of an acid of formula VI using the procedures of Janin and Biagani, Tetrahedron, 1993, 39:10305, or Ford et al., J. Med. Chem., 1985, 28:164.

Certain preferred compounds of formula VI may be prepared by

(i) the reaction sequence

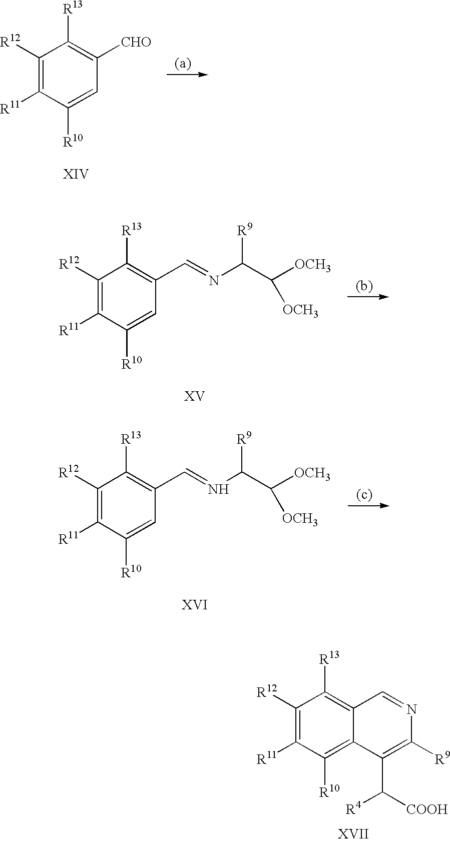

where R⁴, R⁹, R¹⁰, R¹¹, R¹² and R¹³ are as hereinbefore defined. Steps (a) to (c) may be carried out in a known manner, e.g., using the procedure of Dyke et al., Tetrahedron 1968, 24:1467, or analogously as hereinafter described in the Examples;

(ii) the reaction sequence

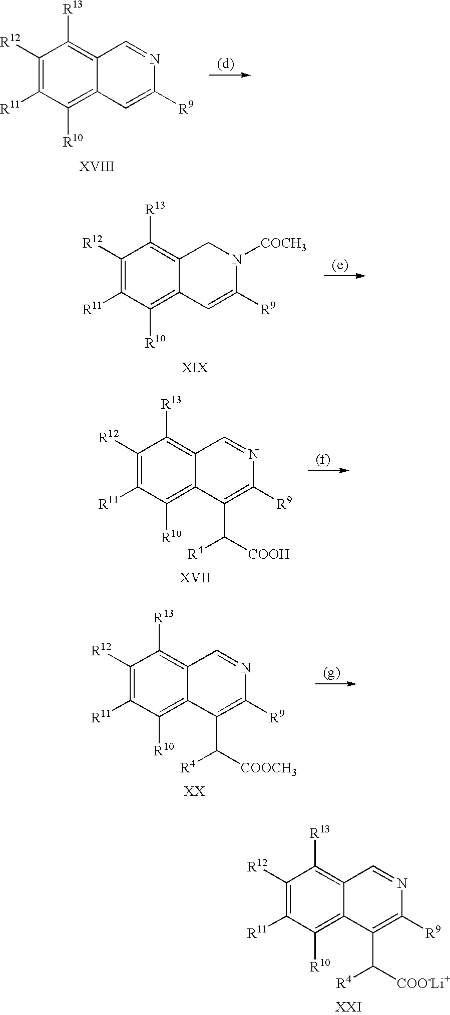

where R⁴, R⁹, R¹⁰, R¹¹, R¹² and R¹³ are as hereinbefore defined. Steps (d) to (g) may be carried out in a known maner, e.g., step (d) using the procedure of Katayama et al., Chem. Pharm. Bull., 1980, 28:2226, step (e) using the procedure of Dyke et al., Tetrahedron, 1968, 24:1467 and steps (f) and (g) using the procedure of March in Advanced Organic Chemistry, 4^{th} Edition, Wiley, NY, 1992:393 and 378, or analogously as hereinafter described in the Examples;

(iii) the reaction

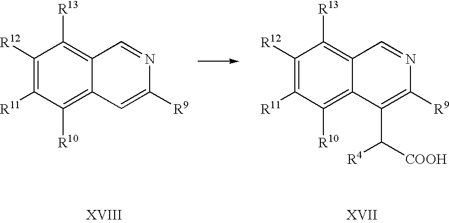

e.g., by treatment of XVIII with a hydride reducing agent, followed by a 2-oxo-carboxylic ester, using the procedure of Minter et al., J. Org. Chem., 1988, 53:2653, or analogously as described hereinafter in the Examples;

(iv) for the preparation of compounds of formula VI in which R⁵ is a 4-isoquinolinyl group substituted in the 1-position, the reaction sequence

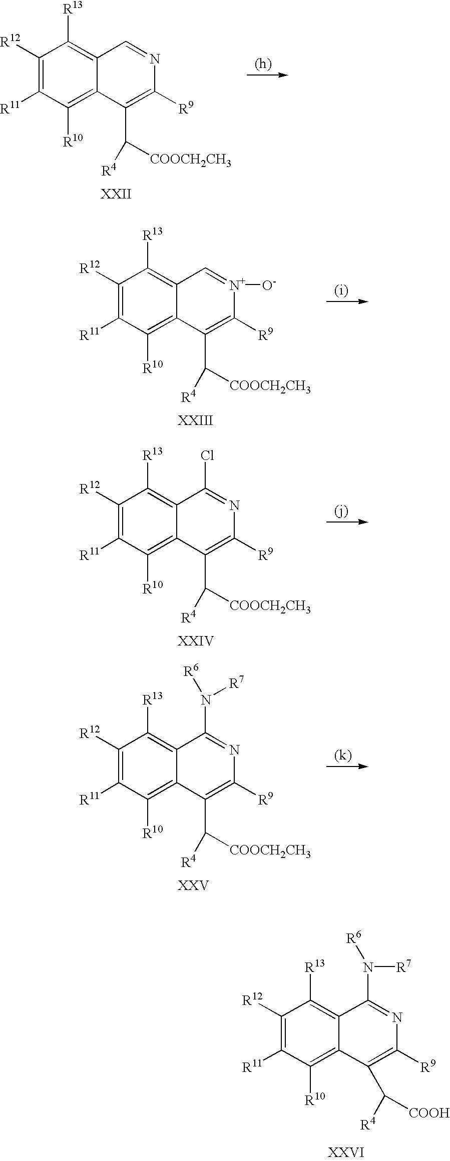

where R⁴, R⁶, R⁷, R⁹, R¹⁰, R¹¹, R¹² and R¹³ are as hereinbefore defined. Steps (h) to (k) may be carried out in a known manner, e.g., steps (h) to (j) using the procedure of Janin and Biagni, Tetrahedron, 1993, 39:10305 and step (k) using the procedure of March, Advanced Organic Chemistry, 4^{th} Edition, NY, 1992:378 or analogously as hereinafter described in the Examples;

(v) for the preparation of compounds of formula VI in which R⁵ is an isoquinolinyl group of formula III in which R⁸ is cyano, the reaction sequence

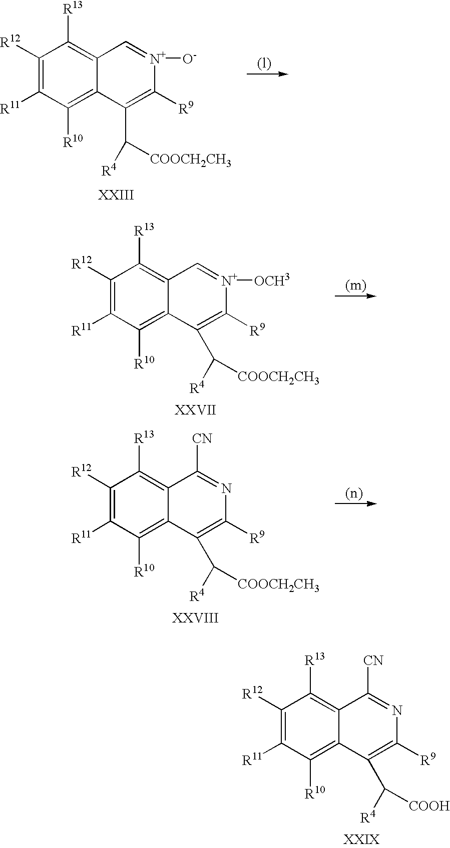

where R⁴, R⁹, R¹⁰, R¹¹, R¹² and R¹³ are as hereinbefore defined. Steps (l) to (n) may be carried out in a known manner, e.g., steps (l) and (m) using the procedure of Ford et al., J. Med. Chem., 1985, 28:164 and step (n) using the procedure of March, op. cit., 378;

(vi) for the preparation of compounds of formula VI in which R⁵ is an oxodihydroisoquinolinyl group, the reaction sequence

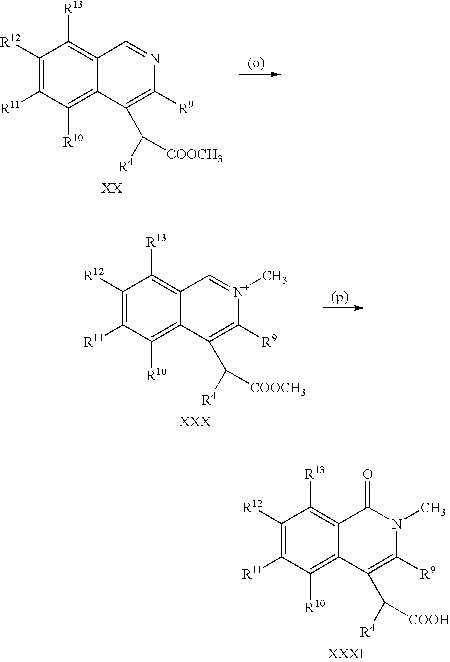

where R⁴, R⁹, R¹⁰, R¹¹, R¹² and R¹³ are as hereinbefore defined. Steps (o) and (p) may be carried out in a known manner, e.g., using the procedure of Holzgrabe, Arch. Pharm., 1988, 321:767, or analogously as hereinafter described in the Examples;

(vii) for preparation of compounds of formula VI where R⁵ is a quinolinyl group, the reaction

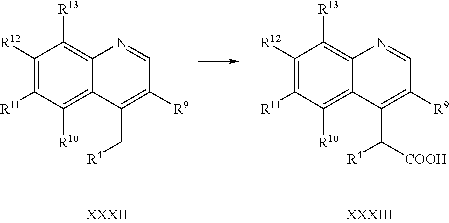

where R⁴, R⁹, R¹⁰, R¹¹, R¹² and R¹³ are as hereinbefore defined, which may be carried out in a known manner, e.g., by treatment with a strong base, preferably an alkali metal dialkylamide, such as lithium diisopropylamide, followed by treatment with carbon dioxide, e.g., using the procedure of using Brown and Curless, Tetrahedron Lett., 1986, 27:6005, or analogously as hereinafter described in the Examples.

(viii) for the preparation of compounds of formula VI where R⁵ is a 4-isoquinolinyl group,

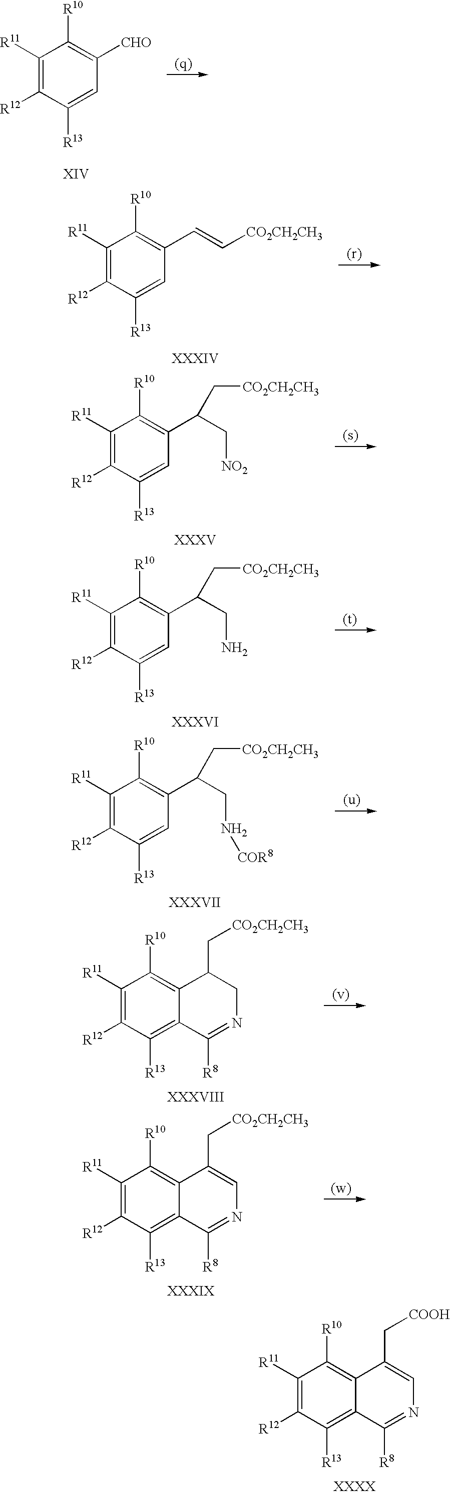

where R⁸, R¹⁰, R¹¹, R¹² and R¹³ are as hereinbefore defined. Steps (q) to (w) may be carried out in a known manner; e.g., step (q) by treatment with a carboxyethyltriarylphosphonium ylid, preferably carboxyethyltriphenylphosphonium ylid in an organic solvent, preferably an ether or hydrocarbon, especially toluene, at sub-ambient, elevated or, preferably, ambient temperature; step (r) by treatment with nitromethane in the presence of an inorganic or, preferably, amine base, especially tetramethylguanidine, for example, in the presence of a solvent or, preferably, in the absence of a solvent, at sub-ambient, ambient or, preferably, elevated temperature, e.g., 60-80° C.; step (s) by treatment with a reducing agent, preferably a tin (II) salt, especially tin (II) chloride hydrate, in an aqueous or, preferably, organic solvent, preferably an alcohol, such as ethanol, at sub-ambient, ambient or, preferably, elevated temperature, e.g., under reflux; step (t) by treatment with an acid halide or anhydride, preferably the acid chloride, of the acid R⁸COOH, at elevated or, preferably, sub-ambient or ambient temperature, e.g., 0° C. to ambient temperature, in an aqueous or, preferably, organic solvent, especially a chlorinated solvent, such as dichloromethane, preferably in the presence of a base, especially an amine, such as triethylamine; step (u) by treatment with a phosphorus (V) halide or oxyhalide, preferably phosphorous pentachloride or phosphorus oxychloride, preferably in an organic solvent, such as a hydrocarbon or nitrile, especially acetonitrile, preferably at ambient or, especially, elevated temperature, e.g., under reflux; step (v) by treatment with a noble metal, preferably palladium, catalyst, preferably in an organic solvent, especially a hydrocarbon, such as decalin, preferably at elevated temperature, e.g., under reflux; step (w) by treatment with an alkali metal hydroxide, preferably lithium or sodium hydroxide, in organic, aqueous or mixed organic-aqueous solvent, preferably THF-water, at sub-ambient, elevated or, preferably, ambient temperature; specific methods for steps (q) to (w) being as hereinafter described in the Examples.

(ix) the reaction sequence

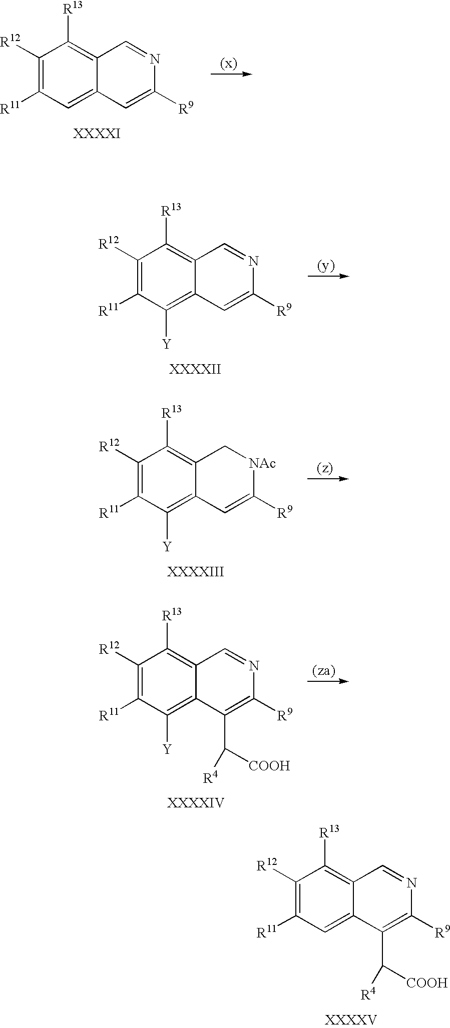

where R⁹, R¹¹, R¹² and R¹³ are as hereinbefore defined, Ac is an acyl group, and Y is halogen. Steps (x) to (za) may be effected in a known manner, e.g., step (x) by reaction with a halogenation agent, e.g. bromine or a N-halosuccinimide, preferably N-chlorosuccinimide, e.g., as described in March, op.cit., 531; step (y) by reaction with a reducing agent, e.g., a metal hydride, in the presence of an acylating agent, e.g., acetic anhydride, e.g., as described in Katayama et al., op.cit; step (z) by reaction with a 2-oxocarboxylic acid, preferably glyoxylic acid, in the presence of a mineral acid, e.g., as described in Dyke et al., Tetrahedron, 1968, 24:1467; and step (za) by treatment with a reducing agent, e.g., as described in March et al., op.cit, 566; or analogously as described hereinafter in the Examples.

Certain compounds of formula V are novel, including Intermediates 1-10 as described hereinafter. Certain compounds of formula VI are novel, including Intermediates 20-48 as described hereinafter.

Process variant (b) may be carried out in a known manner, for example, by reacting a compound of formula I where R³ is hydrogen with an appropriate alkylating agent, preferably an alkyl iodide or dialkyl sulfate, such as a compound of formula R³I or (R³)₂SO₄ where R³ is C₁-C₄-alkyl. The reaction may be conducted in the presence of an inorganic or organic base, for example, in an aqueous, organic or mixed aqueous-organic solvent. Alkylation may be carried out at sub-ambient temperature or, more conveniently, at ambient or elevated temperature. Preferred bases are alkali metal carbonates. Preferred solvents are organic dipolar aprotic solvents, especially N,N-dimethylformamide.

Process variant (c) may be effected using known sulfonylation procedures, e.g., in the presence of an organic or inorganic base, preferably a tertiary organic base, such as pyridine. The reaction temperature may be sub-ambient, ambient or, preferably, elevated. Preferred procedures are as hereinafter described in the Examples.

Process variant (d) may be effected using known procedures, e.g., by treating a compound of formula I wherein R² is alkenyl with a hydroborating agent, followed by oxidative basic work-up. Hydroboration may be carried out at sub-ambient or, more conveniently, at ambient or elevated, temperature. Preferred hydroborating agents are dialkylboranes, such as 9-borabicyclo[2.2.0]nonane, which are preferably reacted under reflux. Oxidative work-up is preferably conducted with hydrogen peroxide and an alkali metal hydroxide, preferably sodium hydroxide. The work-up temperature is preferably 40-60° C.

Process variant (e) may be carried out using conventional esterification procedures, e.g., by reacting the compound of formula I wherein R² is hydroxy with a carboxylic acid or halide thereof, preferably an acid chloride, in the presence of an organic or inorganic base, at sub-ambient or, preferably, ambient or elevated (e.g., 40-60° C.) temperature. Preferred bases are organic tertiary bases, such as pyridine.

Process variant (f) may be carried out using known procedures for conversion of acylamino into amino, e.g., by treatment with a mineral acid, such as sulphuric or, preferably, hydrochloric acid. The reaction is preferably carried out in a mixed aqueous-organic solvent, such as aqueous ethanol. The reaction temperature is conveniently ambient or, preferably, elevated temperature, especially reflux temperature.

Process variant (g) may be effected using known dealkylation methods, e.g., by reaction with HBr or Hi, usually at elevated temperature, preferably by heating with concentrated hydrobromic acid, e.g., as hereinafter described in the Examples.

Process variant (h) may be effected using known halogenation procedures, e.g., by reaction with bromine or chlorine in a solvent, such as acetic acid. The reaction is conveniently carried at ambient temperature, e.g., as hereinafter described in the Examples.

Process variant (i) may be effected using known procedures for the Simmons Smith reaction, e.g., by reaction with diethyl zinc and chloroiodomethane. The reaction is usually carried out in an organic solvent, preferably a halohydrocarbon. The reaction is suitably carried out at ambient temperature, e.g., as hereinafter described in the Examples.

Compounds of formula I in free form may be converted into salt form, and vice versa, in a conventional manner. The compounds in free or salt form can be obtained in the form of hydrates or solvates containing a solvent used for crystallization. The compounds of formula I in free or salt form can be recovered from reaction mixtures in a conventional manner. Isomer mixtures can be separated into individual isomers, e.g., enantiomers, in a conventional manner, e.g., by fractional crystallization.

Furthermore agents of the invention have an appropriate duration of action and many have a rapid onset of action. The inhibiting properties of agents of the invention may be demonstrated in the following test procedure:

PDE5 Assay: A 10 mM solution of a test compound in DMSO is diluted 100-fold with aqueous 20% v/v DMSO to give a 100 μM stock solution, which is further diluted with aqueous 20% v/v DMSO to give ten solutions having concentrations from 10 to 0.00051 μM. 10 μL of each of these solutions is transferred to a selected well of a 96-well Optiplate microtitre plate (e.g., Packard). To determine total binding, 10 μL of aqueous 20% v/v DMSO is added to other selected wells. To determine non-specific binding, a 10 mM solution of sildenafil in 100% DMSO is diluted 20-fold with aqueous 20% v/v DMSO and 10 μL of the resulting solution is added to further selected wells of the Optiplate plate. To all wells containing test compound solution, aqueous DMSO or sildenafil solution is added 80 μL of Assay Mix, prepared by mixing PDE Assay Buffer (2 mL), an aqueous solution of bovine serum albumin (BSA) containing 5 mg BSA/mL (2 mL), an aqueous 75 μM solution of cGMP sodium salt (0.2 mL), 3H-cGMP (e.g., Amersham, 10 μL) and distilled water (11.8 mL). (The PDE Assay Buffer is prepared by dissolving Tris-base (7.56 g) in water (800 mL), adding 1 M aqueous MgCl₂ (10.325 mL) and 0.5 M EDTA (4.25 mL), adjusting the pH to 7.5 with 1N hydrochloric acid and making up to 1 L with water). A solution of human PDE5, partially purified from human platelets (11 μL, containing 0.017 units of PDE5 per mL, where 1 unit hydrolyses 1.0 μmole of 3′,5′-cyclic GMP to 5′-GMP per minute at pH 7.5 at 37° C.), in 20 mM Hepes, pH 7.4, 100 mM sodium chloride, 10% v/v glycerol, 1 mM benzamidine and 2 mM dithiotherietol, is diluted 200-fold, with Enzyme Buffer prepared by adding 0.5 M EDTA (2 mL) to a solution of Tris-Base (1.21 g) in water (800 mL), adjusting the pH to 7.5 with 1N HCl and making up to 1 L with water. The diluted PDE5 solution (10 μL) is added to all wells containing test compound, aqueous DMSO or sildenafil solution. The plate is incubated at room temperature for 1 hour. Fifty (50) μL of a suspension of 500 mg PDE Yttrium silicate SPA beads (e.g., Amersham) in 28 mL water is added to each of the wells and the plate is incubated for a further 20 minutes and then sealed using Top Seal-S (e.g., Packard) according to the manufacturer's instructions. The resulting scintillations are counted using a Canberra Packard Top Count (1 minute per well), as a measure of the extent to which binding of PDE5 to the beads is inhibited. The concentration of test compound at which 50% inhibition of PDE5 binding to the beads occurs (IC₅₀) is determined from concentration-inhibition curves in a conventional manner.

Compounds of the Examples hereinbelow have IC₅₀ values of the order of from 0.0005 μM to 10 μM in the above assay. For example, the compounds of Examples 7, 10, 15, 35, 45, 49, 55, 60, 68 and 70 have IC₅₀ values of 0.007, 0.01, 0.006, 0.010, 0.002, 0.0037, 0.0055, 0.0028, 0.007 and 0.009 μM, respectively, in the above assay.

Intermediates of formula V are prepared as follows:

Intermediate 1: Methallylamine (211 g, 2.97 mol) is added to a solution of concentrated hydrochloric acid (250 mL) in water (1.9 L), followed by portionwise addition of potassium cyanate (240 g, 2.97 mol). The reaction is then heated for 2 hours at 80° C., prior to cooling and evaporation to afford (2-methyl-allyl)-urea (244.5 g), mp 114-115° C. The urea (268 g, 2.35 mol) is added to a solution of cyanoacetic acid (220 g, 2.59 mol) in acetic anhydride (536 mL) and the reaction is heated at 70° C. for 1 hour, cooled to 0° C. and diluted with ether. The resultant solid is collected by filtration, washed with ether, suspended in water (2.2 L) and heated to 75° C. 2 M aqueous sodium hydroxide solution is then added portionwise over 30 minutes to maintain pH between 8 and 9.5. The reaction is cooled to room temperature, treated with acetic acid (12 mL), further cooled to 10° C. and the resultant solid is collected by filtration, washed with cold water and dried to afford 6-amino-1-(2-methyl-allyl)-1H-pyrimidine-2,4-dione, mp 267-269° C. The uracil (253 g, 1.40 mol) is added to a solution of sodium hydroxide (123 g, 3.07 mol) in water (2.5 L) and allowed to exotherm then cooled to 20° C. Dimethyl sulfate (196 mL, 2.06 mol) is added portionwise over 1 hour. After standing overnight, the reaction is cooled to 5° C. and the solid collected by filtration to give 6-amino-3-methyl-1-(2-methyl-allyl)-1H-pyrimidine-2,4-dione, mp 162-163° C. The methyluracil (165 g, 0.85 mol) is suspended in water (1.55 L) and concentrated hydrochloric acid (72 mL). A solution of sodium nitrite (58.4 g, 0.85 mol) in water (117 mL) is then added dropwise over 30 minutes and the reaction is stirred at 20° C. for 3 hours. The solid is collected by filtration, washed successively with water, methanol and ether to afford 6-amino-3-methyl-1-(2-methyl-allyl)-5-nitroso-1H-pyrimidine-2,4-dione, mp 213° C. (dec). The nitrosouracil (190 g, 0.85 mol) is suspended in water (950 mL), heated to 85° C. and sodium dithionite (85%, 347.2 g, 1.69 mol) is added portionwise. After cooling to room temperature, the solid is collected by filtration to afford 5,6-diamino-3-methyl-1-(2-methyl-allyl)-1H-pyrimidine-2,4-dione, mp 152-153° C.

Intermediate 2: Using the general procedure for Intermediate 1, (3-nitrobenzyl)-urea (J. Med. Chem., 1996, 39:1924) is converted into 6-amino-3-methyl-1-(3-nitro-benzyl)-1H-pyrimidine-2,4-dione, [M-H]⁻ 275. A suspension of this compound (4.88 g, 17.7 mmol) and 10% Pd/C (0.484 g) in ethanol (200 mL) is hydrogenated at 1 atmosphere for 1.5 hours. The reaction mixture is filtered through a celite plug and evaporated to give 6-amino-1-(3-amino-benzyl)-3-methyl-1H-pyrimidine-2,4-dione acetic acid salt [M-H]⁻ 245. Acetic anhydride (1.85 mL, 19.57 mmol) is added to a cooled (0° C.) suspension of 6-amino-1-(3-amino-benzyl)-3-methyl-1H-pyrimidine-2,4-dione acetic acid salt (5.01 g, 16.35 mmol) in pyridine (50 mL). The reaction mixture is warmed to room temperature, stirred for 6 hours and the solvent evaporated. The residue is triturated with water and the solid collected by filtration and dried to afford N-[3-(6-amino-3-methyl-2,4-dioxo-3,4-dihydro-2H-pyrimidin-1-ylmethyl)-phenyl]-acetamide, ¹H NMR (400 MHz, DMSO): δ: 2.00 (s 3H), 3.09 (s 3H), 4.72 (s 1H), 5.02 (s 2H), 6.75 (s 2H), 6.88 (d J 6 1H), 7.25 (t J 6 1H), 7.30 (s 1H), 7.55 (d J 6 1H). Using the general procedure for Intermediate 1, this compound is converted into N-[3-(5,6-diamino-3-methyl-2,4-dioxo-3,4-dihydro-2H-pyrimidin-1-ylmethyl)-phenyl]-acetamide [MH]⁺ 304.

Intermediate 3: Using the general procedure for Intermediate 1, (4-nitro-benzyl)-urea (J. Med. Chem., 1996, 39:1924) is converted into 6-amino-3-methyl-1-(4-nitro-benzyl)-1H-pyrimidine-2,4-dione, [MH]⁺ 277. A solution of calcium chloride (4.94 g, 45 mmol) in water (100 mL) is added to a solution of 6-amino-3-methyl-1-(4-nitro-benzyl)-1H-pyrimidine-2,4-dione (19.08 g, 69.0 mmol) in acetic acid (300 mL). Zinc dust (58.8 g, 900 mmol) is then added portionwise with external cooling. The reaction is stirred at room temperature for 1.5 hours, filtered through a celite plug and washed successively with ethanol and acetic acid. Evaporation of the combined filtrate and washings affords 6-amino-1-(4-amino-benzyl)-3-methyl-1H-pyrimidine-2,4-dione acetic acid salt, [M-3H]⁻ 243. Acetic anhydride (7.2 mL, 76.0 mmol) is added to a cooled (0° C.) suspension of 6-amino-1-(4-amino-benzyl)-3-methyl-1H-pyrimidine-2,4-dione acetic acid salt (17.0 g, 69.0 mmol) in pyridine (260 mL). The reaction mixture is warmed to room temperature, stirred for 6 hours and the solvent evaporated. The residue is triturated with water and the solid collected by filtration and dried to afford N-[4-(6-amino-3-methyl-2,4-dioxo-3,4-dihydro-2H-pyrimidin-1-ylmethyl)-phenyl]-acetamide, [MH]⁺ 289. Using the general procedure for Intermediate 1, this compound is converted into N-[4-(5,6-diamino-3-methyl-2,4-dioxo-3,4-dihydro-2H-pyrimidin-1-ylmethyl)-phenyl]-acetamide, [MH]⁺ 304.

Intermediate 4: To a cooled (0° C.) slurry of 6-chloromethyluracil (2.0 g, 12 mmol) in THF/dioxan (1:1, 16 mL) is added 2-pyridyldiphenylphosphine (3.60 g, 13.7 mmol) and cyclobutanemethanol (1.3 mL, 13.8 mmol), followed by di-t-butylazodicarboxylate (3.15 g, 13.7 mmol). The reaction is stirred overnight at ambient temperature, treated with 4 M HCl in dioxan (15 mL) and evaporated. The residue is taken up in dichloromethane, washed with 3.5 M HCl, dried over magnesium sulfate and evaporated. The crude product is purified by flash chromatography (100:1 dichloromethane-methanol elution) to afford 6-chloro-1-cyclobutylmethyl-3-methyl-1H-pyrimidine-2,4-dione, ¹H NMR (400 MHz, CDCl₃) δ 1.70-2.0 (m 6H), 2.60 (m 1H), 3.20 (s 3H), 4.00 (d J 7 2H), 5.78 (s 1H), which is dissolved in n-butanol (50 mL). Veratrylamine (4 mL, 26.5 mmol) is added and the reaction heated to reflux for 16 hours. The solvent is evaporated and the residue taken into dichloromethane, washed with 1 M aqueous HCl, dried over magnesium sulfate and evaporated. The crude product is purified by flash chromatography (50:1 dichloromethane-methanol elution) to afford 1-cyclobutylmethyl-6-(3,4-dimethoxy-benzylamino)-3-methyl-1H-pyrimidine-2,4-dione,

¹H NMR (400 MHz, CDCl₃) d 1.60-1.80 (m 4H), 1.80-2.00 (m 2H), 2.50 (m 1H), 3.21 (s 3H), 3.80 (s 6H), 3.85 (d J 7 2H), 4.11 (d J 5 1H), 4.25 (m 1H), 4.84 (s 1H), 6.74 (s 1H), 6.80 (s 2H), which is dissolved in formic acid (50 mL) and Pd black (0.26 g) added. The reaction is heated at 40° C. for 21 hours, filtered through Celite, evaporated and purified by preparative HPLC to afford 6-amino-1-cyclobutylmethyl-3-methyl-1H-pyrimidine-2,4-dione, M⁺ 0.209, which is converted using the general procedure for Intermediate 1 into 5,6-diamino-1-cyclobutylmethyl-3-methyl-1H-pyrimidine-2,4-dione, HPLC retention time 0.17 minutes (30%-95% acetonitrile water gradient in 4 minutes).

Intermediate 5: 5,6-Diamino-3-methyl-1-(tetrahydro-furan-2-ylmethyl)-1H-pyrimidine-2,4-dione, mp 115-116° C., is prepared from (tetrahydro-furan-2-ylmethyl)-urea (Collect. Czech. Chem. Commun., 1972, 37:2786) using the general procedure for Intermediate 1.

Intermediate 6: 5,6-Diamino-3-methyl-1-(2-methyl-butyl)-1H-pyrimidine-2,4-dione, mp 163-165° C., is prepared using the general procedure for Intermediate 1.

Intermediate 7: 5,6-Diamino-1-hexyl-3-methyl-1H-pyrimidine-2,4-dione, is prepared from 6-amino-1-hexyl-1H-pyrimidine-2,4-dione (J. Med. Chem., 1993, 36:1465) using the general procedure for Intermediate 1, HPLC retention time (0-95% acetonitrile water gradient over 8 minutes) 6.01 minutes.

Intermediate 8: 5,6-Diamino-1-(3,4-dimethoxy-benzyl)-3-methyl-1H-pyrimidine-2,4-dione, [M-H]⁻ 305, is prepared from (3,4-dimethoxy-benzyl)-urea (Farmaco, Ed. Sci., 1977, 32:813) using the general procedure for Intermediate 1.

Intermediate 9: 5,6-Diamino-1-benzo[1,3]dioxol-5-ylmethyl-3-methyl-1H-pyrimidine-2,4-dione, mp 183-186° C., is prepared using the general procedure for Intermediate 1.

Intermediate 10: 5,6-Diamino-1-(2,4-dichloro-benzyl)-3-methyl-1H-pyrimidine-2,4-dione, is prepared using the general procedure for Intermediate 1, ¹H NMR (400 MHz DMSO-d6) δ 3.16 (s 3H), 5.05 (s 2H), 6.18 (s 2H), 6.82 (d J 9 1H), 7.38 (d J 9 1H), 7.62 (s 1H).

Other Intermediates of formula V are prepared according to literature references as indicated below:

| No. | R¹ | R² | Reference |
|---|---|---|---|
| 11 | CH₃ | (CH₃)₂CHCH₂ | (1) |
| 12 | H | CH₃ | (2) |
| 13 | CH₃ | CH₂═CHCH₂ | (3) |
| 14 | CH₃ | 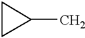 | (1) |
| 15 | CH₃ | (CH₃)₃CCH₂ | (1) |
| 16 | (CH₃)₂CHCH₂ | (CH₃)₂CHCH₂ | (1) |
| 17 | CH₃ | 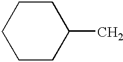 | (1) |
| 18 | H | CH₃CH₂CH₂ | (2) |
| 19 | CH₃ | 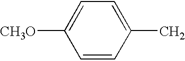 | (4) |

References:
(1) Eur. J. Med. Chem. 1990, 25, 653
(2) J. Med. Chem. 1996, 39, 2
(3) FR 2 531 085
(4) Eur. J. Med. Chem. -Chim. Ther. 1974, 9, 313

Intermediates of formula VI are prepared as follows:

Intermediate 20: A mixture of 3-(3,4-dimethoxy-phenyl)-5-nitro-pentanoic acid ethyl ester (J. Med. Chem, 1989, 32:1450) (0.50 g, 1.68 mmol) and tin(II)chloride dihydrate (1.90 g, 8.4 mmol) in ethanol (10 mL) is heated to reflux for 2 hours, cooled to ambient temperature and evaporated. The crude product is taken into dichloromethane (15 mL), cooled to 0° C. and triethylamine (5 mL) added, followed by 3,5-dimethoxybenzoyl chloride (0.404 g, 2.02 mmol). The reaction is stirred at ambient temperature overnight and then evaporated, taken into ethyl acetate, washed with water and dried over sodium sulfate. Evaporation and purification by flash column chromatography (1:1 hexane-ethyl acetate elution) affords 4-(3,5-dimethoxy-benzoylamino)-3-(3,4-dimethoxy-phenyl)-butyric acid ethyl ester, [MH]⁺ 432. This intermediate (0.200 g, 0.46 mmol) is taken into acetonitrile (8 mL) and phosphorus oxychloride (0.211 g, 1.38 mmol) added, prior to heating at reflux for 3 hours. After evaporation of the solvent, the residue is taken into ethyl acetate, washed with saturated aqueous sodium carbonate, dried over sodium sulfate and evaporated to afford [1-(3,5-dimethoxy-phenyl)-6,7-dimethoxy-3,4-dihydro-isoquinolin-4-yl]-acetic acid ethyl ester, [MH]⁺ 414. This intermediate (0.50 g, 1.21 mmol) is dissolved in decalin (10 mL) and 10% Pd/C (50 mg) added. The reaction is heated at 190° C. for 2.5 hours, then cooled to ambient temperature and diluted with dichloromethane. After filtration through Celite, the combined filtrate and washings are evaporated to afford [1-(3,5-dimethoxy-phenyl)-6,7-dimethoxy-isoquinolin-4-yl]-acetic acid ethyl ester, ¹H NMR (400 MHz, CDCl₃) δ 1.18 (t J 7 3H), 3.78 (s 6H), 3.80 (s 3H), 3.90 (s 2H), 3.99 (s 3H), 4.10 (q J 7 2H), 6.50 (d J 0.5 1H), 6.75 (d J 0.5 2H), 7.20 (s 1H), 7.36 (s 1H), 8.40 (s 1H). This intermediate (0.30 g, 0.73 mmol) is dissolved in methanol (10 mL), 1 M aqueous lithium hydroxide (0.80 mL, 0.80 mmol) is added and the reaction stirred overnight at ambient temperature. After evaporation of the methanol, pH of the residual solution is adjusted to 7 with 1 M aqueous HCl and the resultant solid collected by filtration and dried to afford [1-(3,5-dimethoxy-phenyl)-6,7-dimethoxy-isoquinolin-4-yl]-acetic acid.

Intermediate 21: A mixture of 3-isopropoxy-4-methoxy-benzaldehyde (3.9 g, 20 mmol) and (ethoxycarbonylmethylene)triphenylphosphorane (6.96 g, 20 mmol) in toluene (100 mL) is heated at reflux for 2 hours, cooled to ambient temperature and evaporated. The crude product is taken in to dichloromethane and eluted through a pad of silica to afford (E)-3-(3-isopropoxy-4-methoxy-phenyl)-acrylic acid ethyl ester, TLC R_{f} 0.70 (1:1 hexane-ethyl acetate). This intermediate is dissolved in nitromethane (10 mL), 1,1,3,3-tetramethylguanidine (0.5 mL) is added and the reaction heated at 70° C. for 36 hours. The solvent is evaporated, the residue is taken into ethyl acetate and washed with 2N aqueous HCl, water and brine. After drying over sodium sulfate and evaporation, the crude product is purified by flash column chromatography (4:1 hexane-ethyl acetate elution) to afford 3-(3-isopropoxy-4-methoxy-phenyl)-4-nitro-butyric acid ethyl ester, ¹H NMR (400 MHz, CDCl₃) δ 1.20 (t J 7 3H), 1.38 (d J 7 6H), 2.75 (d J 6 2H), 3.90 (m 1H), 4.10 (m 2H), 4.48-4.78 (m 3H), 6.75-6.86 (m 3H). This intermediate is converted into [1-(3,5-diisopropoxy-phenyl)-6-isopropoxy-7-methoxy-isoquinolin-4-yl]-acetic acid using the general procedure for Intermediate 20. Characterised as the ethyl ester, [MH]⁺ 496.

Intermediate 22: A solution of 3-ethoxy-4-methoxybenzaldehyde (3.6 g, 20 mmol) in ethanol (15 mL) is added to 2,2-dimethoxyethylamine (21 mmol) and the mixture heated at reflux for 2 hours. After cooling to room temperature, sodium borohydride (0.794 g, 21 mmol) is added and the mixture stirred at room temperature overnight. Ethanol is removed by evaporation and water added, followed by extraction with ethyl acetate. The organic extracts are combined, washed with water, brine, dried over magnesium sulfate and evaporated to give (2,2-dimethoxy-ethyl)-(3-ethoxy-4-methoxy-benzyl)-amine, [MH]⁺ 270. The intermediate (2.70 g, 10 mmol) is suspended in 6N HCl (50 mL), glyoxylic acid (0.88 g, 12 mmol) is added and the mixture heated for 1 hour at 100° C. After cooling to room temperature, methanol (30 mL) is added and the mixture filtered and characterised as the methyl ester M⁺ 276. The filtrate is treated with lithium hydroxide (10 mmol) in THF-methanol-water overnight. After evaporation of the solvents, the crude product is partitioned between water and dichloromethane. The aqueous phase is washed with dichloromethane and evaporated to dryness to afford (7-ethoxy-6-methoxy-isoquinolin-4-yl)-acetic acid lithium salt which is used for xanthine formation without further characterisation.

Intermediate 23: A solution of (6,7-dimethoxy-isoquinolin-4-yl)-acetic acid ethyl ester (Tetrahedron, 1973, 29:3881) (1.668 g, 6.07 mmol) in chloroform (20 mL) is treated portionwise with m-chloroperoxybenzoic acid (1.153 g, 6.67 mmol) for 5 hours. The reaction mixture is washed with saturated sodium bicarbonate and brine, dried over MgSO₄ and evaporated to afford (6,7-dimethoxy-2-oxy-isoquinolin-4-yl)-acetic acid ethyl ester 1.71 g, 96%). The entire product is dissolved in chloroform (30 mL), added to POCl₃ (3 mL, 32.3 mmol) and heated at reflux for 2 hours. After evaporation, dichloromethane and ice water are added and the mixture is basified with aqueous ammonia. The aqueous phase is further extracted with dichloromethane, the combined organic phases are washed with brine, dried over magnesium sulfate and evaporated to afford (1-chloro-6,7-dimethoxy-isoquinolin-4-yl)-acetic acid ethyl ester. The chloro ester derivative (0.50 g, 1.6 mmol) is suspended in 2 M sodium hydroxide (15 mL). Ethanol (5 mL) is added and the solution stirred at room temperature for 2 hours and the solvent evaporated. Adjustment to pH 2 with concentrated hydrochloric acid affords a solid which is collected by filtration and dried to afford (1-chloro-6,7-dimethoxy-isoquinolin-4-yl)-acetic acid, ¹H NMR (400 MHz, DMSO-d₆) δ 3.92 (s 3H), 3.96 (s 3H), 4.02 (s 2H), 7.31 (s 1H), 7.44 (s 1H), 8.04 (s 1H).

Intermediate 24: (2,2-Dimethoxy-ethyl)-(3-methoxy-benzyl)-amine (Tetrahedron, 1973, 29, 3881) is treated with pyruvic acid according to the general procedure for Intermediate 22 to afford 2-(7-methoxy-isoquinolin-4-yl)-propionic acid hydrochloride salt, mp 174-176° C. Treatment with HCl gas in ethanol affords the corresponding ethyl ester hydrochloride, mp 190-192° C., which is then reacted sequentially with m-chloroperoxybenzoic acid and phosphorus oxychloride as described for Intermediate 23 to provide 2-(1-chloro-7-methoxy-isoquinolin-4-yl)-propionic acid ethyl ester, mp 126-128° C. This intermediate (47 g, 0.16 mol) is dissolved in ethanol (400 mL) and 2N sodium hydroxide (150 mL) added and the mixture heated at 60° C. for one hour, prior to evaporation of the solvent. Crystallisation from acetone affords 2-(1-chloro-7-methoxy-isoquinolin-4-yl)-propionic acid, mp 167-168° C.

Intermediate 25: Dimethyl sulfate (12.7 mL, 0.10 mol) is added portionwise to 2-(7-methoxy-2-oxy-isoquinolin-4-yl)-propionic acid ethyl ester (28 g, 0.10 mol) with an exotherm to 100° C. The reaction is maintained at this temperature for 2 hours, cooled to room temperature and dissolved in water (50 mL). A solution of sodium cyanide (15 g, 0.31 mol) in water (90 mL) is added over 30 minutes with external cooling and the reaction is then stirred at room temperature for 3 hours. The crude product is extracted with chloroform, the chloroform extracts are washed with saturated sodium bicarbonate, brine, dried over sodium sulfate and evaporated. Crystallisation from 3N ethanolic HCl-ether affords 2-(1-cyano-7-methoxy-isoquinolin-4-yl)-propionic acid ethyl ester hydrochloride, mp 89-98° C. This compound is hydrolysed converted to the acid as described for Intermediate 22 and used directly crude for xanthine formation.

Intermediate 26: A solution of sodium triethylborohydride (1 M THF, 12.7 mL, 12.7 mmol) is added dropwise to a solution of isoquinoline (1.64 g, 12.7 mmol) in THF (25 mL). The reaction is stirred at room temperature for 1 hour, prior to dropwise addition of a solution of ethyl glyoxalate (1.43 g, 13.9 mmol) in toluene (previously heated at 110° C. for 1.5 hours). After a further 4 hours at room temperature, the reaction is cooled to 0° C. and sodium hydroxide (0.5 M aqueous solution, 25.4 mL) followed by hydrogen peroxide (30% aqueous solution, 12.7 mL) is added, followed by stirring for 2 hours. The reaction is acidified with 1N HCl, washed with ethyl acetate three times, the aqueous phase is reduced in volume by evaporation and refrigerated overnight. The resultant precipitate is collected by filtration and dried to afford isoquinolin-4-yl-acetic acid hydrochloride salt, MH⁺ 188.

Intermediate 27: Excess morpholine is added to a suspension of (1-chloro-6,7-dimethoxy-isoquinolin-4-yl)-acetic acid ethyl ester (0.200 g, 0.65 mmol) in toluene (1 mL) and the mixture heated to reflux until the starting material is consumed. After evaporation, the residue is partitioned between water and dichloromethane, the organic phase is dried over magnesium sulfate and evaporated. to afford (6,7-dimethoxy-1-morpholin-4-yl-isoquinolin-4-yl)-acetic acid ethyl ester [MH]⁺ 361. The crude ester (0.240 g, 0.66 mmol) is dissolved in ethanol (20 mL), treated with 2 M sodium hydroxide (3 mL) and stirred at room temperature overnight. After adjustment to pH 1 with concentrated hydrochloric acid, the solvent is evaporated and the crude acid used directly for formation of the xanthine derivative.

Intermediate 28: The procedure for Intermediate 27 is repeated, using excess N-methylpiperazine in place of morpholine to afford [6,7-dimethoxy-1-(4-methyl-piperazin-1-yl)-isoquinolin-4-yl]-acetic acid ethyl ester (0.186 g, 38%), ¹H NMR (DMSO-d6) δ 1.19 (t J 7 3H), 2.30 (s 3H), 2.61 (m 4H), 3.10-3.30 (m 4H), 3.92 (s 6H), 3.97 (s 2H), 4.10 (q J 7 2H), 7.19 (s 1H), 7.37 (s 1H), 7.91 (s 1H). The ester (0.186 g, 0.50 mmol) is dissolved in ethanol (20 mL), treated with 2 M sodium hydroxide (3 mL) and stirred at room temperature overnight. After adjustment to pH 1 with concentrated hydrochloric acid, the solvent is evaporated and the crude acid used directly for formation of the xanthine derivative.

Intermediate 29: N-Chlorosuccinimide (0.347 g, 2.60 mmol) is added to a solution of 6-methoxyisoquinoline (Synth. Commun., 1999, 29:1617) (0.207 g, 1.30 mmol) in acetic acid (9 mL). The reaction is heated at 50° C. for 3 hours, cooled to ambient temperature, evaporated and partitioned between ethyl acetate and 1 M aqueous sodium hydroxide. The organic phase is washed with water and brine, dried over magnesium sulfate and evaporated afford 5-chloro-6-methoxyisoquinoline, [MH]⁺ 194. A solution of this intermediate (0.175 g, 0.90 mmol) in THF (4.5 mL) and acetic anhydride (0.101 mL, 1.08 mmol) is treated with sodium triacetoxyborohydride (0.229 g, 1.08 mmol) and the reaction is stirred at ambient temperature for 22 hours. The solvent is evaporated, the residue is taken into ethyl acetate, washed with 0.5 M aqueous hydrochloric acid, then brine and dried over magnesium sulfate. Evaporation affords 1-(5-chloro-6-methoxy-1H-isoquinolin-2-yl)-ethanone, mp 78-80° C. A suspension of this intermediate (0.150 g, 0.60 mmol) and glyoxylic acid (76 mg, 0.80 mmol) in 6 M aqueous hydrochloric acid (2.8 mL) is heated at 100° C. for 3 hours. After cooling to ambient temperature, the resultant solid is collected by filtration to afford (5-chloro-6-methoxy-isoquinolin-4-yl)-acetic acid, [MH]⁺ 252. A suspension of this intermediate (0.970 g, 3.38 mmol) and ammonium formate (1.05 g, 16.9 mmol) in 1:1 acetic acid-water (25 mL) is treated with 10% Pd/C (0.730 g) and stirred at ambient temperature for 16 hours. After filtration through Celite®, the combined filtrate and washings are evaporated and purified by Soxhlet extraction with acetone to afford (6-methoxy-isoquinolin-4-yl)-acetic acid [MH]⁺ 218. Alternatively reduction of (5-chloro-6-methoxy-isoquinolin-4-yl)-acetic acid to afford (6-methoxy-isoquinolin-4-yl)-acetic acid is accomplished by stirring a suspension of (5-chloro-6-methoxy-isoquinolin-4-yl)-acetic acid (20 g, 69.4 mmol) in 1 M sodium hydroxide solution (400 mL) for 20 minutes, filtering off the resultant salt and then treating with hydgrogen gas in the presence of 10% Pd/C (1.4 g) at atmospheric pressure for 2.25 hours. The resulting suspension is filtered through glass wool and celite, washing with water (50 mL). The solution is then cooled in an ice water bath and slowly (30 minutes) neutralised and then acidified with 5 M hydrochloric acid (80 mL). A suspension forms and further crystalisation is encouraged by standing at 5° C. for 20 hours. The resulting crystals are removed by filtration and washed with ice cold ethanol (25 mL) drying under reduced pressure gives (6-methoxy-isoquinolin-4-yl)-acetic acid.

Intermediate 30: Glyoxylic acid (1.37 g, 9.28 mmol) is added to mixture of 1-(6-chloro-1H-isoquinolin-2-yl)-ethanone (J. Org. Chem., 1980, 45:1950) (1.44 g, 5.90 mmol) in 6N HCl (24 mL). The reaction is heated at 100° C. for 3 hours, cooled to RT, washed with ether and evaporated to 10 mL volume. After overnight refrigeration, the solid is collected by filtration and dried to afford (6-chloro-isoquinolin-4-yl)-acetic acid hydrochloride, ¹H NMR (400 MHz, DMSO) δ 4.45 (s 2H), 8.18 (d J 9 1H), 8.52 (s 1H), 8.70 (d J 8 1H), 8.83 (s 1H), 9.96 (s 1H).

Intermediate 31: Sodium borohydride (1.12 g, 29.6 mmol) is added portionwise to a cooled (0° C.) solution of 6-bromoisoquinoline (J. Chem. Soc. Perkin Trans., 1998, 2:437) (1.544 g, 7.42 mmol) in acetic acid (10 mL) and acetic anhydride (3 mL). After heating at 60° C. for 4 hours, the mixture is cooled, evaporated and diluted with water. After adjustment to pH 10 with potassium carbonate and extraction with ethyl acetate, the combined organic phases are washed twice with 0.5N HCl and brine, then dried over sodium sulphate. Evaporation affords 1-(6-bromo-1H-isoquinolin-2-yl)-ethanone, MH⁺ 253. Glyoxylic acid (0.812 g, 8.80 mmol) is added to mixture of 1-(6-bromo-1H-isoquinolin-2-yl)-ethanone (1.50 g, 5.90 mmol) in 6N HCl (20 mL). The reaction is heated at 100° C. for 2 hours, cooled to RT and washed with ethyl acetate. After evaporation, the residue is taken into methanol (20 mL), concentrated sulphuric acid (10 drops) is added and the mixture heated at reflux for 14 hours. After partial evaporation of the solvent, the resultant solid is collected by filtration, washed with methanol and dried to afford (6-bromo-isoquinolin-4-yl)-acetic acid methyl ester hydrochloride, MH⁺ 281. Lithium hydroxide hydrate (8.5 mg, 0.20 mmol) is added to a cooled (0° C.) solution of (6-bromo-isoquinolin-4-yl)-acetic acid methyl ester (50 mg, 0.18 mmol) in 3:1 THF-water (3 mL). After 1 hour the solvent is evaporated to afford (6-bromo-isoquinolin-4-yl)-acetic acid lithium salt, [MH]⁺ 266.

Intermediate 32: Trimethylsilylacetylene (0.17 mL, 1.23 mmol) is added to a suspension of (6-bromo-isoquinolin-4-yl)-acetic acid methyl ester (0.325 g, 1.03 mmol) in DMF (1.75 mL) and triethylamine (10 mL), follwed by copper (I) iodide (40 mg, 0.20 mmol) and (Ph₃P)₂PdCl₂ (73 mg, 0.10 mmol). The reaction is heated at 45° C. for 40 minutes, cooled to ambient temperature and diluted with ethyl acetate. After washing with water and brine, the organic phase is dried over magnesium sulfate, evaporated and purified by flash column chromatography (1:1 ethyl acetate-hexane elution) to afford (6-trimethylsilanylethynyl-isoquinolin-4-yl)-acetic acid methyl ester, [MH]⁺ 298. This intermediate (0.221 g, 0.74 mmol) is dissolved in methanol (7.5 mL) and treated with potassium carbonate (75 mg, 0.54 mmol). The reaction is stirred for 30 minutes at ambient temperature, evaporated and purified by flash chromatography (5:1 dichloromethane-methanol elution) to afford (6-ethynyl-isoquinolin-4-yl)-acetic acid, [MH]⁺ 212.

Intermediate 33: Bromine (0.211 mL, 6.28 mmol) in dichloromethane (10 mL) is added to a cooled (0° C.) solution of 6-methoxyisoquinoline (Synth. Commun., 1999, 29:1617) and the reaction is stirred at ambient temperature for 20 hours. After pouring into 1 M aqueous sodium hydroxide, the organic phase is washed with brine, dried over magnesium sulfate and evaporated. The crude product is purified by flash column chromatography (20:1 dichloromethane-methanol elution) to afford 5-bromo-6-methoxyisoquinoline, [MH]⁺ 240. This material is then converted according to the procedure for Intermediate 29 into (5-bromo-6-methoxy-isoquinolin-4-yl)-acetic acid [MH]⁺ 298.

Intermediate 34: [1-(3,5-Diisopropoxy-phenyl)-6,7-dimethoxy-isoquinolin-4-yl]-acetic acid is prepared using the general procedure for Intermediate 20, ¹H NMR (400 MHz CDCl₃) δ 1.25 (d J 6 1 2H), 3.78 (s 3H), 3.86 (s 2H), 3.92 (s 3H), 6.46 (d J 0.5 1H), 6.65 (d J 0.5 2H), 7.20 (s 2H), 8.30 (s 1H).

The following are prepared analogously to Intermediate 21:

Intermediate 35: 1-(3,5-Dimethoxy-phenyl)-6-isopropoxy-7-methoxy-isoquinolin-4-y]-acetic acid, [MH]⁺ 412.

Intermediate 36: (1-.tert.-Butyl-6-isopropoxy-7-methoxy-isoquinolin-4-yl)-acetic acid,

¹H NMR (400 MHz, CDCl₃) δ 1.32 (d J 7 6H), 1.52 (s 9H), 3.80 (s 2H), 3.90 (s 3H), 4.75 (heptet J 7 1H), 7.28 (s 1H), 7.66 (s 1H), 8.08 (s 1H).

Intermediate 37: (6-Isopropoxy-1-isopropyl-7-methoxy-isoquinolin-4-yl)-acetic acid, [MH]⁺⁻318.

The following are prepared analogously to Intermediate 20:

Intermediate 38: (6,7-Dimethoxy-1-methyl-isoquinolin-4-yl)-acetic acid, [MH]⁺ 262. Intermediate 39: (1-tert.-Butyl-6,7-dimethoxy-isoquinolin-4-yl)-acetic acid, ¹H NMR (400 MHz, CDCl₃) d 1.75 (s 9H), 3.95 (s 6H), 4.04 (s 2H), 7.28 (s 1H), 7.75 (s 1H), 8.66 (s 1H).

Intermediate 40: (1-Isopropyl-6,7-dimethoxy-isoquinolin-4-yl)-acetic acid, characterised as the ethyl ester, ¹H NMR (400 MHz, CDCl₃) δ 1.25 (t J 7 3H), 1.45 (d J 7 3H), 3.82 (heptet J 7 1H), 3.90 (s 2H), 3.08 (s 2H), 4.15 (q J 7 2H), 7.28 (s 1H), 7.48 (s 1H), 8.30 (s 1H).

Intermediate 41: 2-(7-Methoxy-1-morpholin-4-yl-isoquinolin-4-yl)-propionic acid mp 225-227° C., is prepared according to the procedure for Intermediate 27.

The following are prepared analogously to Intermediate 22:

Intermediate 42: [MH]⁺ 332 (7-Hydroxy-6-methoxy-isoquinolin-4-yl)-acetic acid lithium salt, via (3-benzyloxy-4-methoxy-benzyl)-(2,2-dimethoxy-ethyl)-amine.

Intermediate 43: (6,7-Dimethoxy-3-methyl-isoquinolin-4-yl)-acetic acid, ¹H NMR (400 MHz, DMSO) δ 2.50 (s 3H), 3.91 (s 3H), 3.93 (s 3H), 4.02 (s 2H), 7.30 (s 1H), 7.43 (s 1H), 8.30 (s 1H).

Intermediate 44: (6-Ethoxy-7-methoxy-isoquinolin-4-yl)-acetic acid, 3 M⁺ 261.

The following are prepared analogously to Intermediate 22, using pyruvic acid in place of glyoxylic acid:

Intermediate 45: 2-(6-Ethoxy-7-methoxy-isoquinolin-4-yl)-propionic acid lithium salt, characterised as the methyl ester, M⁺ 290.

Intermediate 46: 2-(7-Ethoxy-6-methoxy-isoquinolin-4-yl)-propionic acid lithium salt, characterised as the methyl ester, M⁺ 290.

Intermediate 47: 2-(6,7-dimethoxy-isoquinolin-4-yl)-propionic acid, characterised as the methyl ester, M⁺ 276.

Intermediate 48: 8-Fluoro-6-methoxy-isoquinolin-4-yl)-acetic acid, is prepared according to the procedure for Intermediate 31 and characterised as the methyl ester, [MH]⁺ 250.

Intermediate 49: (6,7-Dimethoxy-isoquinolin-4-yl)-acetic acid; and

Intermediate 50: [1,3]dioxolo[4,5-.g.]isoquinolin-8-yl-acetic acid, are prepared as described in Dyke et al., Tetrahedron, 1968, 24:1467.

Intermediate 51: (7-Methoxy-isoquinolin-4-yl)-acetic acid is prepared according to Dyke et al., Tetrahedron, 1973, 29:3881.

Intermediate 52: 2,2-Dimethoxyethylamine (13.85 mL, 0.13 mol) is added to a solution of 3-fluoro-4-methoxybenzaldehyde (20 g, 0.13 mol) in toluene (200 mL). The resulting solution is flushed with nitrogen gas and then heated overnight under reflux in a Dean-Stark apparatus. The solvent is then removed under reduced pressure to yield (2,2-dimethoxy-ethyl)-[1-(3-fluoro-4-methoxy-phenyl)-methylidene]-amine. This intermediate (31 g, 0.13 mol) is dissolved in ethylacetate and acetic anhydride (13.1 g, 0.13 mol) added. Platinum oxide (0.3 g) is then added, under a blanket of nitrogen, and the resulting mixture is stirred under a hydrogen atmosphere until uptake is complete. Filtration, washing with saturated aqueous NaHCO₃ (3×100 mL), brine and water, drying over MgSO₄ and concentration then gives N-(2,2-dimethoxy-ethyl)-N-(3-fluoro-4-methoxy-benzyl)-acetamide. This intermediate (38.9 g, ca 0.13 mol) is dissolved in anhydrous CH₂Cl₂ and then added slowly over 20 minutes to a stirred mixture of A1C1₃ (90 g) and CH₂Cl₂ under an atmosphere of nitrogen. The total volume of CH₂Cl₂ is 250 mL. The mixture is stirred for a further 10 minutes at room temperature and is then cooled with an ice bath during the addition of aqueous 40% NaOH. The mixture is further diluted with water (250 mL), filtered through glass wool, the organic phase separated and the aqueous phase further extracted with CH₂Cl₂ (2×200 mL). Drying over MgSO₄ and evaporation under reduced pressure yields a crude oil which is purified by flash silica chromatography (eluant: 1% methanol in CH₂Cl₂) to give as one of the products 1-(7-fluoro-6-methoxy-1H-isoquinolin-2-yl)-ethanone. This intermediate (0.60 g, 2.7 mmol) is mixed with glyoxylic acid (0.325 g, 3.5 mmol) and water (10 mL) and the resulting mixture is stirred at room temperature for 20 minutes. Concentrated hydrochloric acid (10 mL) is then added and the mixture heated to reflux for 1 hour. Concentration and purification by preparative HPLC gives (7-fluoro-6-methoxy-isoquinolin-4-yl|)-acetic acid, [MH]⁺ 236.

The following are prepared analogously to Intermediate 20:

Intermediate 53: (1-Methyl-6-methoxy-isoquinolin-4-yl)-acetic acid.

Intermediate 54: (6-Isopropoxy-1-methyl-isoquinolin-4-yl)-acetic acid.

Intermediate 55: (6-Ethoxy-1-methyl-isoquinolin-4-yl)-acetic acid.

Intermediate 56: A solution of (6-bromo-isoquinolin-4-yl)-acetic acid methyl ester (52 mg, 0.19 mmol), prepared as described as an intermediate for Intermediate 31, in DMF (3 mL) is added to zinc dicyanide (26 mg, 0.22 mmol) under a nitrogen atmosphere. To the resulting mixture is added 1,1′-bis(diphenylphosphino)ferrocene (15 mg) and tris(dibenzylideneacetone)dipalladium (0) (8 mg) and the resulting mixture stirred at 120° C. for 22 hours. The solution is cooled and diluted with chloroform (30 mL) and washed with water (2×20 mL) followed by brine (20 mL). Further chlorofrom is added (40 mL) and the olution dried over MgSO₄, filtered and concentrated. Repetitive flash silica column chromatography (eluants 40:1 CH₂Cl₂:methanol, then 50:1 CH₂Cl₂:methanol) gives (6-cyano-isoquinolin-4-yl)-acetic acid methyl ester [MH]⁺ 227. This intermediate is saponified by treatment with LiOH in 3:1 THF/water. The resulting mixture is partially evaporated to remove the THF, diluted to 10 mL with water then washed with ethyl acetate. The aqeous phase is then neutralised with 1 M hydrochloric acid (to pH 4-5) and exhaustively extracted with ethylacetate. The organic phase is dried over MgSO₄, filtered and concentrated to give (6-cyano-isoquinolin-4-yl)-acetic acid M⁺ 212.

Intermediate 57: (5-Chloro-6-methoxy-isoquinolin-4-yl)-acetic acid is prepared as described in the procedure for Intermediate 29.

Intermediate 58: To a solution of (6-trimethylsilanylethynyl-isoquinolin-4-yl)-acetic acid methyl ester, as prepared for Intermediate 32 (0.19 g, 0.64 mmol), in anhydrous methanol (7 mL) is added K₂CO₃ (72 mg) and the resulting mixture stirred for 1 hour. Additional K₂CO₃ (11 mg) is then added and stirring continued for 30 minutes. The mixture is then neutralised with glacial acetic acid and concentrated. Purification by flash silica column chromatography (ethylacetate/hexane 1:1) gives (6-ethynyl-isoquinolin-4-yl)-acetic acid methyl ester M⁺ 225. This intermediate (79 mg, 0.35 mmol) is dissolved in methanol under an inert atmosphere and 10% Pd on carbon (79 mg) added. The resulting suspension is stirred vigorously under an atmosphere of gaseous hydrogen. After 90 minutes, filtration, washing with methanol and concentration give (6-ethyl-isoquinolin-4-yl)-acetic acid methyl ester M⁺ 229. To a solution of this intermediate (68 mg, 0.30 mmol) in THF/methanol/water (3:1:1, 3.5 mL) is added LiOH (12.5 mg) and the mixture stirred for 20 hours at room temperature. Concentration under reduced pressure gives lithium (6-ethyl-isoquinolin-4-yl)-acetate M⁺ 221.

Intermediate 59: A solution of Intermediate 29 (0.5 g, 2.3 mmol) is suspended in aqueous 48% HBr (10 mL) and then heated at 100° C. for 48 hours. Further aqueous 48% HBr (10 mL) is then added and heating continued at 100° C. for an additional 24 hours. The reaction mixture is cooled to 5° C. for 4 hours and the resulting solid seperated by filtration. Washing with water and drying under high vacumn at 50° C. gives (6-hydroxy-isoquinolin-4-yl)-acetic acid hydrobromide [MH]⁺ 204.4. This intermediate (0.15 g, 0.53 mmol) is suspended in DMF (2 mL) and K₂CO₃ (0.22 g, 1.58 mmol) added followed by ethyliodide (0.085 mL, 1.06 mmol) and the resulting mixture stirred at room temperature for 2 hours. Concentration and purification by flash silica column chromatography (eluant: CH₂Cl₂/methanol 10:1) gives (6-ethoxy-isoquinolin-4-yl)-acetic acid ethyl ester [MH]⁺ 260. This intermediate (25 mg, 0.11 mmol) is dissolved in water (1 mL) and LiOH added (5 mg, 0.11 mmol). The resulting mixture is stirred for 30 minutes at room temperature. Acidification with minimumn 6N HCl and concentration gives crude (6-ethoxy-isoquinolin-4-yl)-acetic acid.

### EXAMPLES 1-70

Compounds of formula I which are also of formula

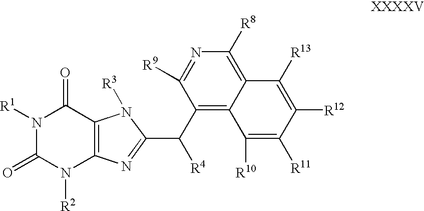

where R¹ to R⁴ and R⁸ to R¹³ are as hereinbefore defined, in free or salt form, and their methods of preparation are shown in the following table, the methods being described hereinafter. R³ is H in all Examples except No. 44, where it is CH₃. R⁴ is H in all examples except Nos. 25-27 and 41-43, where it is CH₃. R⁹ is H in all Examples except No. 29, where it is CH₃. R¹⁰ is H in all Examples except No. 57, where it is Br and No. 75 where it is Cl. R¹³ is H in all Examples except No. 56 where it is F, and Nos. 65 and 66, where it is Br.

| | | | | | | m/z | | | |
|---|---|---|---|---|---|---|---|---|---|
| Ex. No. | R¹ | R² | R⁸ | R¹¹ | R¹² | MH+ | MH− | Mthd | Reacting Intermediates |
| 1 | CH₃ | (CH₃)₂CHCH₂ | 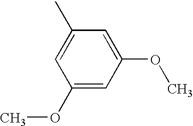 | OCH₃ | OCH₃ | 560 | | A | 11 + 20 |
| 2 | CH₃ | (CH₃)₂CHCH₂ | 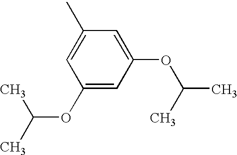 | OCH₃ | OCH₃ | 616 | | A | 11 + 34 |
| 3 | CH₃ | (CH₃)₂CHCH₂ | 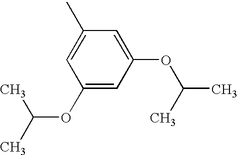 | OCH(CH₃)₂ | OCH₃ | 644 | | A | 11 + 21 |
| 4 | CH₃ | (CH₃)₂CHCH₂ | 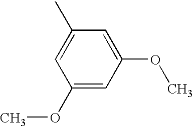 | OCH(CH₃)₂ | OCH₃ | 588 | | A | 11 + 35 |
| 5 | CH₃ | (CH₃)₂CHCH₂ | (CH₃)₃C | OCH(CH₃)₂ | OCH₃ | 508 | | A | 11 + 36 |
| 6 | CH₃ | (CH₃)₂CHCH₂ | (CH₃)₂CH | OCH(CH₃)₂ | OCH₃ | 494 | | A | 11 + 37 |
| 7 | CH₃ | (CH₃)₂CHCH₂ | CH₃ | OCH₃ | OCH₃ | 437 | | A | 11 + 38 |
| | | | | | | (M+) | | | |
| 8 | CH₃ | (CH₃)₂CHCH₂ | (CH₃)₃C | OCH₃ | OCH₃ | 480 | | A | 11 + 39 |
| 9 | CH₃ | (CH₃)₂CHCH₂ | (CH₃)₂CH | OCH₃ | OCH₃ | 465 | | A | 11 + 40 |
| | | | | | | (M+) | | | |
| 10 | CH₃ | (CH₃)₂CHCH₂ | H | OCH₃ | OCH₃ | 424 | | C | 11 + 49 |
| 11 | CH₃ | 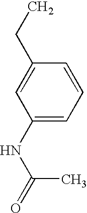 | H | OCH₃ | OCH₃ | | 513 | A | 2 + 49 |
| 12 | H | CH₃ | H | OCH₃ | OCH₃ | | | C | 12 + 49 |
| 13 | CH₃ | CH₂═CHCH₂ | H | OCH₃ | OCH₃ | | | C | 13 + 49 |
| 14 | CH₃ | 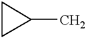 | H | OCH₃ | OCH₃ | | | C | 14 + 49 |
| 15 | CH₃ | (CH₃)₃CCH₂ | H | OCH₃ | OCH₃ | | | C | 15 + 49 |
| 16 | (CH₃)₂CHCH₂ | (CH₃)₂CHCH₂ | H | OCH₃ | OCH₃ | | | C | 16 + 49 |
| 17 | CH₃ | 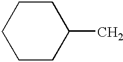 | H | OCH₃ | OCH₃ | | | C | 17 + 49 |
| 18 | CH₃ | CH₂═C(CH₃)CH₂ | H | OCH₃ | OCH₃ | | | C | 1 + 49 |
| 19 | CH₃ | 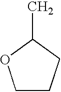 | H | OCH₃ | OCH₃ | | | C | 5 + 49 |
| 20 | CH₃ | 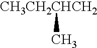 | H | OCH₃ | OCH₃ | | | C | 6 + 49 |
| 21 | H | CH₃CH₂CH₂ | H | OCH₃ | OCH₃ | | | C | 18 + 49 |
| 22 | CH₃ | 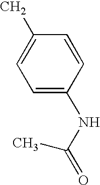 | H | OCH₃ | OCH₃ | | 515 | C | 3 + 49 |
| 23 | CH₃ | (CH₃)₂CHCH₂ | H | 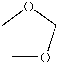 | | | | C | 11 + 50 |
| 24 | CH₃ | (CH₃)₂CHCH₂ | H | H | OCH₃ | 394 | | C | 11 + 51 |
| 25 | CH₃ | (CH₃)₂CHCH₂ | Cl | H | OCH₃ | 442 | | C | 11 + 24 |
| 26 | CH₃ | (CH₃)₂CHCH₂ | CN | H | OCH₃ | 433 | | C | 11 + 25 |
| 27 | CH₃ | (CH₃)₂CHCH₂ | 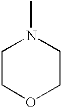 | H | OCH₃ | 493 | | C | 11 + 41 |
| 28 | CH₃ | (CH₃)₂CHCH₂ | H | OCH₃ | OH | 410.7 | | D | 11 + 42 |
| 29 | CH₃ | (CH₃)₂CHCH₂ | H | OCH₃ | OCH₃ | 438.6 | | D | 11 + 43 |
| 30 | CH₃ | CH₃(CH₂)₅ | H | OCH₃ | OCH₃ | 452.8 | | D | 7 + 49 |
| 31 | CH₃ | 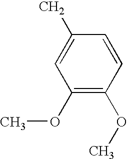 | H | OCH₃ | OCH₃ | 518.4 | | D | 8 + 49 |
| 32 | CH₃ | 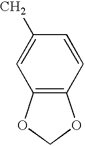 | H | OCH₃ | OCH₃ | 502.4 | | D | 9 + 49 |
| 33 | CH₃ | 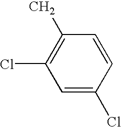 | H | OCH₃ | OCH₃ | 527.8 | | D | 10 + 49 |
| 34 | CH₃ | 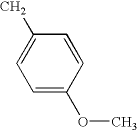 | H | OCH₃ | OCH₃ | 487.9 | | D | 19 + 49 |
| 35 | CH₃ | (CH₃)₂CHCH₂ | Cl | OCH₃ | OCH₃ | 458.4 | | D | 11 + 23 |
| 36 | CH₃ | (CH₃)₂CHCH₂ | H | H | H | 364 | | C | 11 + 26 |
| 37 | CH₃ | (CH₃)₂CHCH₂ | H | OCH₂CH₃ | OCH₃ | 438 | | C | 11 + 44 |
| 38 | CH₃ | (CH₃)₂CHCH₂ | 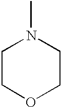 | OCH₃ | OCH₃ | 509.1 | | D | 11 + 27 |
| 39 | CH₃ | (CH₃)₂CHCH₂ | 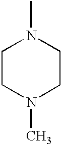 | OCH₃ | OCH₃ | 522.02 | | D | 11 + 28 |
| 40 | CH₃ | (CH₃)₂CHCH₂ | H | OCH₃ | OCH₂CH₃ | 438 | | C | 11 + 22 |
| 41 | CH₃ | (CH₃)₂CHCH₂ | H | OCH₂CH₃ | OCH₃ | 452 | | C | 11 + 45 |
| 42 | CH₃ | (CH₃)₂CHCH₂ | H | OCH₃ | OCH₂CH₃ | 452 | | C | 11 + 46 |
| 43 | CH₃ | (CH₃)₂CHCH₂ | H | OCH₃ | OCH₃ | 438 | | C | 11 + 47 |
| 44 | CH₃ | (CH₃)₂CHCH₂ | H | OCH₃ | OCH₃ | 438 | | J | — |
| 45 | CH₃ | (CH₃)₂CHCH₂ | H | OCH₃ | H | 394 | | B2 | 11 + 29 |
| 46 | CH₃ | 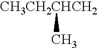 | H | OCH₃ | H | 408 | | C | 6 + 29 |
| 47 | CH₃ | (CH₃)₃CCH₂ | H | Cl | H | | 410 | C | 15 + 30 |
| 48 | CH₃ | (CH₃)₂CHCH₂ | H | Cl | H | | 396 | C | 11 + 30 |
| 49 | CH₃ | 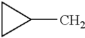 | H | OCH₃ | H | | | C | 14 + 29 |
| 50 | CH₃ | 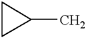 | H | Cl | H | | 394 | C | 14 + 30 |
| 51 | CH₃ | 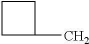 | H | OCH₃ | OCH₃ | 436.6 | 434.5 | B1 | 4 + 49 |
| 52 | CH₃ | CH₂═C(CH₃)CH₂ | H | OCH₃ | H | 392 | 390 | C | 1 + 29 |
| 53 | CH₃ | (CH₃)₂CHCH₂ | H | Br | H | 441 | | C | 11 + 31 |
| 54 | CH₃ | (CH₃)₃CCH₂ | H | OCH₃ | H | 408 | | C | 15 + 29 |
| 55 | CH₃ | (CH₃)₂CHCH₂ | H | C≡CH | H | 388 | | B1 | 11 + 32 |
| 56 | CH₃ | (CH₃)₂CHCH₂ | H | OCH₃ | H | 412 | | C | 11 + 48 |
| 57 | CH₃ | (CH₃)₂CHCH₂ | H | OCH₃ | H | 473 | | C | 11 + 33 |
| 58 | CH₃ | 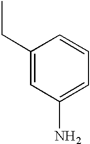 | H | OCH₃ | OCH₃ | | | E | — |
| 59 | CH₃ | 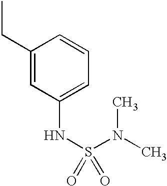 | H | OCH₃ | OCH₃ | | | F | — |
| 60 | CH₃ | 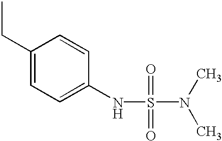 | H | OCH₃ | OCH₃ | 580 | | F | — |
| 61 | CH₃ | 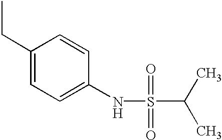 | H | OCH₃ | OCH₃ | 578 (M+) | | F | — |
| 62 | CH₃ | 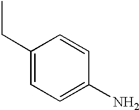 | H | OCH₃ | OCH₃ | 473 | | E | — |
| 63 | CH₃ | (CH₃)₂CHCH₂ | H | H | OH | 379 | | G | — |
| 64 | CH₃ | (CH₃)₂CHCH₂ | H | OH | OH | 396 | | G | — |
| 65 | CH₃ | (CH₃)₂CHCH₂ | H | OH | OH | 474 | | H | — |
| 66 | CH₃ | (CH₃)₂CHCH₂ | H | H | OH | | | H | — |
| 67 | CH₃ | HO(CH₂)₃ | H | OCH₃ | OCH₃ | 426 | | I | — |
| 68 | CH₃ | 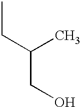 | H | OCH₃ | OCH₃ | 440 | | I | — |
| 69 | CH₃ | 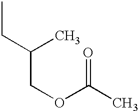 | H | OCH₃ | OCH₃ | 482 | | K | — |
| 70 | CH₃ | 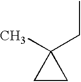 | H | OCH₃ | OCH₃ | 436 | | L | — |
| 71 | CH₃ | 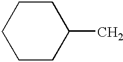 | H | OCH₃ | H | 434 | | B1 | 17 + 29 |
| 72 | CH₃ | 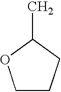 | H | OCH₃ | H | 422 | | B1 | 5 + 29 |
| 73 | CH₃ | (CH₃)₂CHCH₂ | H | OCH₃ | F | 412 | 410 | B1 | 11 + 52 |
| 74 | CH₃ | (CH₃)₂CHCH₂ | H | CO₂H | H | 408 | | C | 11 + 56 |
| 75 | CH₃ | (CH₃)₂CHCH₂ | H | OCH₃ | H | 428 | | B1 | 11 + 57 |
| 76 | CH₃ | (CH₃)₂CHCH₂ | H | CN | H | 389 | | M | |
| 77 | CH₃ | (CH₃)₂CHCH₂ | H | CH₂CH₃ | H | 392 | | B1 | 11 + 58 |
| 78 | CH₃ | (CH₃)₂CHCH₂ | H | OCH₂CH₃ | H | 408.54 | | B1 | 11 + 59 |
| 79 | CH₃ | 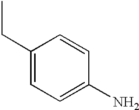 | H | OCH₃ | H | 443.44 | | B1 + E | 3 + 29 |
| 80 | CH₃ | 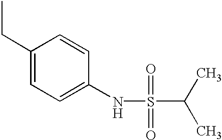 | H | OCH₃ | H | 549 | 547 | F | |
| 81 | CH₃ | 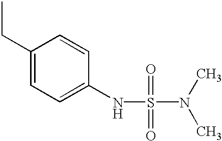 | H | OCH₃ | H | 549 M⁺ | | F | |
| 82 | CH₃ | (CH₃)₂CHCH₂ | N(CH₂)₃ | OCH₃ | OCH₃ | 467 | 465 | N | 11 + 23 |
| 83 | CH₃ | (CH₃)₂CHCH₂ | 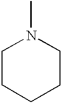 | OCH₃ | OCH₃ | 507 | 505 | O | 11 + 23 |
| 83 | CH₃ | (CH₃)₂CHCH₂ | CH₃ | OCH₃ | H | 408 | 406 | A | 11 + 53 |
| 84 | CH₃ | (CH₃)₂CHCH₂ | CH₃ | OCH(CH₃)₂ | H | M⁺ 435 | | A | 11 + 54 |
| 85 | CH₃ | (CH₃)₂CHCH₂ | CH₃ | OCH₂CH₃ | H | 422 | 420 | A | 11 + 55 |

Method A: 1-(3-Dimethylaminopropyl)-3-ethylcarbodiimide hydrochloride (0.201 g, 1.30 mmol) is added to 5,6-diamino-1-isobutyl-3-methyl-1H-pyrimidine-2,4-dione (0.223 g, 1.05 mmol) and (6,7-dimethoxy-1-methyl-isoquinolin-4-yl)-acetic acid (0.25 g, 0.96 mmol) in methanol (5 mL) and water (1 mL) and the mixture is stirred at ambient temperature for 16 hours. The methanol is evaporated and the resultant solid collected by filtration, taken into methanol (5 mL) and 5 M aqueous sodium hydroxide (0.5 mL) is added. The reaction is heated to reflux for 1 hour, cooled to ambient temperature and evaporated. The residue is dissolved in water and extracted with dichloromethane, the combined organic extracts are dried over sodium sulfate and evaporated to afford 8-(6,7-dimethoxy-1-methyl-isoquinolin-4-ylmethyl)-3-isobutyl-1-methyl-3,7-dihydro-purine-2,6-dione, M⁺ 437.

Method B1: (6-Ethynyl-isoquinolin-4-yl)-acetic acid (58 mg, 0.28 mmol) is dissolved in DMF (1 mL) and O-(7-azabenzotriazo-1-yl)-N,N,N′,N′-tetramethyluronium hexafluorophosphate (0.125 g, 0.33 mmol) and Hunig's base (0.180 mL, 1.03 mmol) are added, followed by a solution of 5,6-diamino-1-isobutyl-3-methyl-1H-pyrimidine-2,4-dione (58 mg, 0.28 mmol) in DMF (0.7 mL). The reaction is stirred at room temperature for 2 hours. The solvent is evaporated and the residue purified by flash column chromatography (30:1 dichloromethane-methanol elution). The intermediate is dissolved in methanol (2 mL) and water (2.75 mL) added, followed by 4 M aqueous sodium hydroxide (0.25 mL). The reaction is heated at 40° C. for 2 hours, then stirrred for 16 hours at ambient temperature. The solvent is evaporated and the crude product purified by flash column chromatography (30:1 dichloromethane-methanol elution) to afford 8-(6-ethynyl-isoquinolin-4-ylmethyl)-3-isobutyl-1-methyl-3,7-dihydro-purine-2,6-dione, [MH]⁺ 388.

Method B2: A suspension of (6-methoxy-isoquinolin-4-yl)-acetic acid (3.5 g, 13.82 mmol) in acetonitrile (70 mL) is treated sequentially with Hunig's base (6.15 mL, 36 mmol), O-(7-benzotriazo-1-yl)-N,N,N′,N′-tetramethyluronium hexafluorophosphate (6.29 g, 16.6 mmol) and 5,6-diamino-1-isobutyl-3-methyl-1H-pyrimidine-2,4-dione (3.22 g, 15.2 mmol) while the solution is stirred at room temperature. The reaction is stirred at ambient temperature for 2 hours, prior to evaporation of the solvent. The residue is tritruated with ethyl acetate (50 mL) filtered and washed with ethyl acetate and then dried at 50° C. under reduced pressure. The resulting intermediate is suspended in a mixture of methanol (30 mL) and 4 M aqueous sodium hydroxide (60 mL) and heated at 80° C. for 45 minutes. This suspension is neutralised with acetic acid and cooled to 0-5° C. overnight. The resultant solid is collected by filtration, and washed with methanol/water 1:9 (30 mL) followed by methanol (30 mL). Drying under high vacuum at 50° C. affords 3-isobutyl-8-(6-methoxy-isoquinolin-4-ylmethyl)-1-methyl-3,7-dihydro-purine-2,6-dione, [MH]⁺ 394.5.

Method C: 1-(3-Dimethylaminopropyl)-3-ethylcarbodiimide hydrochloride (5.6 M aqueous solution, 0.33 mL, 1.85 mmol) is added to a suspension of 5,6-diamino-1-isobutyl-3-methyl-1H-pyrimidine-2,4-dione (0.327 g, 1.54 mmol), (1-chloro-6,7-dimethoxy-isoquinolin-4-yl)-acetic acid (0.414 g, 1.54 mmol) and 1-hydroxybenzotriazole (0.251 g, 1.85 mmol) in CH₂Cl₂ (2 mL). Water (2 mL) is added, the biphasic mixture is shaken for 18 hours and the the resultant solid is collected by filtration. This intermediate is suspended in methanol (10 mL), 4 M aqueous NaOH (5 mL) is added and the mixture heated to reflux for 4 hours. After evaporation of the methanol, the residue is acidified to pH 2 with concentrated hydrochloric acid and the resultant solid collected by filtration and purified by preparative HPLC to afford 8-(1-chloro-6,7-dimethoxy-isoquinolin-4-ylmethyl)-3-isobutyl-1-methyl-3,7-dihydro-purine-2,6-dione hydrochloride, [MH]⁺ 458.

Method D: 1-(3-Dimethylaminopropyl)-3-ethylcarbodiimide (20.6 mL, 0.11 mol) is added to a mixture of 5,6-diamino-1-isobutyl-3-methyl-1H-pyrimidine-2,4-dione (20 g, 0.094 mol), (5,6-dimethoxy-isoquinolin-4-yl)-acetic acid (26.7 g, 0.094 mol), and 1-hydroxybenzotriazole (19.2 g, 0.142 mol) in 1:1 dichloromethane-water (400 mL). The reaction is stirred at ambient temperature for 4.5 hours and the resultant solid collected by filtration. Slurrying in water (500 mL), filtration and washing with water (250 mL) followed by drying, further tritruation with methanol and drying gives an intermediate together with slightly less pure material from concentration of the methanol triturate. The intermediate (16.08 g) is dissolved in water (100 mL) and methanol (100 mL) followed by the addition of 4 M aqueous sodium hydroxide (56 mL) and the resultant solution is heated at 70° C. over night. After cooling to ambient temperature, the methanol is evaporated and the residue acidified to pH 1 with concentrated hydrochloric acid. The resultant hydrochloride salt is collected by filtration and dried. The product is then be converted to the free base by treatment with aqueous sodium hydroxide to pH 11 and washing with water to afford 3-isobutyl-8-(5,6-dimethoxy-isoquinolin-4-ylmethyl)-1-methyl-3,7-dihydro-purine-2,6-dione, [MH]⁺ 424.6.

Method E: A suspension of the product of Example 11 (72 mg, 0.13 mmol) in 6N HCl (2.5 mL) and ethanol (1.5 mL) is heated to reflux for 5 hours then stood at room temperature overnight. The resultant precipitate is collected by filtration, washed with water and dried to afford 3-(3-amino-benzyl)-8-(6,7-dimethoxy-isoquinolin-4-ylmethyl)-1-methyl-3,7-dihydro-purine-2,6-dione dihydrochloride, ¹H NMR (400 MHz, DMSO) δ: 3.20 (s 3H), 3.95 (s 3H), 4.00 (s 3H), 4.75 (s 2H), 5.15 (s 2H), 7.15 (m 2H), 7.20 (s 1H), 7.30 (t J 6 1H), 7.65 (s 1H), 7.95 (s 1H), 8.50 (s 1H), 9.50 (s 1H), 13.6 (br s 1H).

Method F: The product of Example 58 (37 mg, 0.07 mmol) is suspended in pyridine (1.5 mL) and dimethylsulfamoyl chloride (23 mL, 0.21 mmol) is added. The reaction is heated at 50° C. for 22 hours and the solvent is evaporated. Trituration with water gives a solid which is collected by filtration and dried to afford 3-[3-(N,N-dimethylsulfamoyl)amino-benzyl]-8-(6,7-dimethoxy-isoquinolin-4-ylmethyl)-1-methyl-3,7-dihydro-purine-2,6-dione,

¹H NMR (400 MHz, DMSO) δ 2.64 (s 6H), 3.26 (s 3H), 3.86 (s 3H), 3.98 (s 3H), 4.50 (s 2H), 5.15 (s 2H), 6.98 (d J 6 1H), 7.08 (d J 6 1H), 7.15 (s 1H), 7.22 (t J 6 3H), 7.55 (s 1H), 7.62 (s 1H), 8.38 (s 1H), 9.15 (s 1H), 9.82 (s 1H), 13.60 (s 1H).

Method G: The product of Example 24 (100 mg, 0.25 mmol) is heated at 100° C. in concentrated hydrobromic acid (5 mL) for 36 hours. The solvent is evaporated and the crude product purified by preparative HPLC to afford 8-(7-hydroxy-isoquinolin-4-ylmethyl)-3-isobutyl-1-methyl-3,7-dihydro-purine-2,6-dione, [M]⁺ 379.

Method H: The product of Example 64 (41 mg, 0.09 mmol) is dissolved in acetic acid (2 mL) and treated with bromine in acetic acid (148 mg/mL solution: 100 μL). After 1 hour at room temperature, the solvent is evaporated, the residue dissolved in hot methanol, filtered and evaporated to afford 8-(8-bromo-6,7-dihydroxy-isoquinolin-4-ylmethyl)-3-isobutyl-1-methyl-3,7-dihydro-purine-2,6-dione, M⁺ 474.

Method I: A suspension of the product of Example 13, 3-allyl-8-(6,7-dimethoxy-isoquinolin-4-ylmethyl)-1-methyl-3,7-dihydro-purine-2,6-dione hydrochloride salt (0.760 g, 1.87 mmol), 9-borabicyclo[2.2.0]nonane (0.5 M THF solution, 18.7 mL, 9.35 mmol) and diisopropylethylamine (0.33 mL, 1.89 mmol) in THF (9 mL) is heated to reflux for 2.5 hours. Sodium hydroxide (4 M aqueous solution, 6 ml) and hydrogen peroxide (27.5%, 3 mL) are added sequentially and the reaction heated at 50° C. for 1.5 hours. After evaporation, the crude product is purified by flash chromatography (19:1 CH₂Cl₂-methanol elution) and triturated with water to afford 8-(6,7-dimethoxy-isoquinolin-4-ylmethyl)-3-(3-hydroxy-propyl)-1-methyl-3,7-dihydro-purine-2,6-dione, [MH]⁺ 426.

Method J: Potassium carbonate (48 mg, 0.35 mmol) and iodomethane (0.018 mL, 0.295 mmol) are added to a solution of the product of Example 10, 8-(6,7-dimethoxy-isoquinolin-4-ylmethyl)-3-isobutyl-1-methyl-3,7-dihydro-purine-2,6-dione (0.100 g, 0.24 mmol) in DMF (2 mL). The reaction is stirred overnight and purified by preparative HPLC to afford 8-(6,7-dimethoxy-isoquinolin-4-ylmethyl)-3-isobutyl-1,7-dimethyl-3,7-dihydro-purine-2,6-dione, [MH]⁺ 438.

Method K: A suspension of the product of Example 68, 8-(6,7-dimethoxy-isoquinolin-4-ylmethyl)-3-(3-hydroxy-2-methyl-propyl)-1-methyl-3,7-dihydro-purine-2,6-dione (63 mg, 0.14 mmol) and acetyl chloride (18 mL, 0.25 mmol) in pyridine (1 mL) is heated at 50° C. for 18 hours. After evaporation, flash chromatography (19:1 dichloromethane-methanol elution) affords acetic acid 3-[8-(6,7-dimethoxy-isoquinolin-4-ylmethyl)-1-methyl-2,6-dioxo-1,2,6,7-tetrahydro-purin-3-yl]-2-methyl-propyl ester, [MH]⁺ 482.

Method L: The product of Example 18, 8-(6,7-dimethoxy-isoquinolin-4-ylmethyl)-1-methyl-3-(2-methyl-allyl)-3,7-dihydro-purine-2,6-dione (100 mg, 0.24 mmol) is suspended in 1,2-dichloroethane (30 mL). Diethyl zinc (1 M hexane solution, 1.2 mL, 1.20 mmol) is added, followed by chloroiodomethane (0.174 mL, 0.24 mmol) and the reaction is stirred at ambient temperature for 1 hour, prior to quenching with saturated aqueous NH₄Cl. After extraction with chloroform, the organic phase is washed with water, dried over MgSO₄ and evaporated. Purification by preparative HPLC affords 8-(6,7-dimethoxy-isoquinolin-4-ylmethyl)-1-methyl-3-(1-methyl-cyclopropylmethyl)-3,9-dihydro-purine-2,6-dione, [MH]⁺ 436.

Method M: The product of Example 53, 8-(6-bromo-isoquinolin-4-ylmethyl)-3-isobutyl-1-methyl-3,7-dihydro-purine-2,6-dione (245 mg, 0.554 mmol) is dispersed in a mixture of triethylamine (0.085 mL, 0.61 mmol) and CH₂Cl₂ (4 mL). To the stirred mixture is added dropwise a solution of di-tert butoxycarbonate (133 mg, 0.61 mmol) in CH₂Cl₂ (1 mL); after 2 hours triethylamine (0.170 mL, 1.2 mmol), di-tert butoxycarbonate (130 mg, 0.60 mmol) and DMF (0.3 mL) are added and the mixture is stirred at room temperature for 2.5 days. Concentration, partitioning between water and hexane, sonication filtration, re-concentration followed by purification by flash silica column chromatography (eluant 19:1 CH₂Cl₂: methanol) gives 8-(6-bromo-isoquinolin-4-ylmethyl)-3-isobutyl-1-methyl-2,6-dioxo-1,2,3,6-tetrahydro-purine-7-carboxylic acid .tert.-butyl ester, [MH]⁺ 543. This intermediate (58 mg, 0.11 mmol) is added to Zn(CN)₂ (15 mg, 0.13 mmol) followed by 1,1′-bis(diphenylphosphino)ferrocene (9 mg), tris(dibenzylideneacetone) dipalladium (0) (5 mg) and anhydrous DMF (2.5 mL) and the resulting mixture stirred at 120° C. for 18 hours and then for a further 24 hours at 150° C. Zn(CN)₂ (57 mg, 0.49 mmol) and anhydrous DMF (1 mL) are then added and the mixture is heated for 2 hours at 155° C. for 2 hours followed by 18 hours at 145° C. 1,1′-Bis(diphenylphosphino)ferrocene (9 mg), tris (dibenzylideneacetone)dipalladium (0) (9 mg) are then added and the reaction is heated for a further 6 hours at 145° C. Concentration, tritruation with water, filtration, washing with 1:1 saturated NaHCO₃/water, followed by extraction with CH₂Cl₂ and 1:1 methanol:CH₂Cl₂ and repetitive flash silica column chromatography (eluants 10:1 CH₂Cl₂:methanol then 20:1 CH₂Cl₂: methanol) gives 4-(3-isobutyl-1-methyl-2,6-dioxo-2,3,6,7-tetrahydro-1H-purin-8-ylmethyl)-isoquinoline-6-carbonitrile, [MH]⁺ 389.

Method N: 1-(3-Dimethylaminopropyl)-3-ethylcarbodiimide hydrochloride (0.29 g, 1.9 mmol) is added to 5,6-diamino-1-isobutyl-3-methyl-1H-pyrimidine-2,4-dione (0.40 g, 1.9 mmol) and (1-chloro-6,7-dimethoxy-isoquinolin-4-yl)-acetic acid (0.39 g, 1.78 mmol) in methanol and water and the mixture is stirred at ambient temperature for 2 hours. The methanol is evaporated and the resultant solid collected by filtration and re-crystallised from ethylacetate/methanol. The resulting solid is heated in a sealed tube (100° C., 8 hours) with 40% aqueous dimethylamine. The mixture is evaporated and extracted with ethylacetate. The ethylacetate solution is then washed with water and brine, dried over sodium sulphate, filtered and concentrated. Further purification by flash silica column chromatography (eluant: ethylacetate/methanol) yields 8-(1-dimethylamino-6,7-dimethoxy-isoquinolin-4-ylmethyl)-3-isobutyl-1-methyl-3,7-dihydro-purine-2,6-dione, MH⁺ 467.

Method O: 1-(3-Dimethylaminopropyl)-3-ethylcarbodiimide hydrochloride (0.29 g, 1.9 mmol) is added to 5,6-diamino-1-isobutyl-3-methyl-1H-pyrimidine-2,4-dione (0.40 g, 1.9 mmol) and (1-chloro-6,7-dimethoxy-isoquinolin-4-yl)-acetic acid (0.39 g, 1.78 mmol) in methanol and water and the mixture is stirred at ambient temperature for 2 hours. The methanol is evaporated and the resultant solid collected by filtration and re-crystallised from ethylacetate/methanol. The resulting solid is heated under reflux with piperidine for 8 hours. The solution is filtered and the resulting solution washed with water and brine, dried over sodium sulphate, filtered and concentrated. Further purification by flash silica column chromatography (eluant: ethylacetate/methanol) yields a solid which is dissolved 20% 1N NaOH/methanol and heated to reflux for 2 hours. Concentration, addition of water and extraction with ethyl acetate gives an organic fraction which is washed with water and brine, dried over Na₂SO₄, filtered and concentrated to give 8-(6,7-dimethoxy-1-piperidin-1-yl-isoquinolin-4-ylmethyl)-3-isobutyl-1-methyl-3,7-dihydro-purine-2,6-dione, MH⁺ 507.

NMR Data for Examples (¹H 400 MHz DMSO-d6)

Example 12: δ 3.25 (s 3H), 3.92 (s 3H), 4.02 (s 3H), 4.65 (s 2H), 7.70 (s 1H), 7.88 (s 1H) 8.45 (s 1H), 9.42 (s 1H), 11.1 (s 1H), 13.60 (s 1H).

Example 13: δ 3.20 (s 3H), 4.95 (s 3H), 4.00 (s 3H), 4.52 (d J 4 2H), 4.70 (s 2H), 5.04 (d J 18 1H), 5.09 (d J 10 1H), 5.88 (m 1H), 7.60 (s 1H), 7.88 (s 1H), 8.46 (s 1H), 9.42 (s 1H), 13.7 (s 1H).

Example 14: δ 0.20-0.40 (m 4H), 1.10-1.30 (m 1H), 3.21 (s 3H), 3.81 (m 2H), 3.98 (s 3H) 4.03 (s 3H), 4.66 (s 2H), 7.65 (s 1H), 7.85 (s 1H), 8.45 (s 1H), 9.39 (s 1H), 13.70 (s 1H).

Example 15: δ 0.82 (s 9H), 3.20 (s 3H), 3.78 (s 2H), 3.99 (s 3H), 4.04 (s 3H), 7.62 (s 1H) 7.90 (s 1H), 8.45 (s 1H), 9.44 (s 1H), 13.60 (s 1H).

Example 16: δ 0.81 (d J 7 12H), 1.98 (m 1H), 2.12 (m 1H), 3.70 (d J 8 2H), 3.78 (d J 7 2H) 3.99 (s 3H), 4.05 (s 3H), 4.70 (s 2H), 7.65 (s 1H), 7.90 (s 1H), 8.46 (s 1H), 9.45 (s 1H), 13.6 (s 1H).

Example 17: δ 0.80-1.10 (m 6H), 1.40-1.60 (m 4H), 1.80 (m 1H), 3.15 (s 1H), 3.76 (d J 8 2H), 3.91 (s 3H), 4.02 (s 3H), 4.68 (s 2H), 7.60 (s 1H), 7.88 (s 1H), 8.44 (s 1H), 13.60 (s 1H).

Example 18: δ 1.69 (s 3H), 3.21 (s 3H), 3.98 (s 3H), 4.01 (s 3H), 4.46 (s 2H), 4.52 (s 1H) 4.68 (s 2H), 4.76 (s 1H), 7.58 (s 1H), 7.84 (s 1H), 8.45 (s 1H), 9.42 (s 1H), 13.60 (s 1H).

Example 19: δ 1.50-1.85 (m 4H), 3.18 (s 3H), 3.50-3.85 (m 4H), 3.95 (s 3H), 4.02 (s 3H), 4.10-4.20 (m 1H), 4.70 (s 2H), 7.75 (s 1H), 7.920 (s 1H), 8.50 (s 1H), 9.50 (s 1H), 13.60 (br s 1H).

Example 20: δ 0.70-0.80 (m 6H), 0.99-1.10 (m 1H), 1.20-1.25 (m 1H), 1.88-2.00 (m 1H) 3.21 (s 3H), 3.64-3.80 (m 2H), 3.95 (s 3H), 4.00 (s 3H), 4.68 (s 2H), 7.60 (s 1H), 7.80 (s 1H), 8.45 (s 1H), 9.42 (s 1H), 13.60 (br s 1H).

Example 21: δ 0.83 (t J 8 3H), 1.63 (sextet J 8 2H), 3.83 (t J 8 2H), 3.99 (s 3H), 4.05 (s 3H) 4.69 (s 2H), 7.64 (s 1H), 7.88 (s 1H), 8.44 (s 1H), 9.42 (s 1H), 11.10 (s 1H), 13.60 (s 1H).

Example 23: δ 0.80 (d J 7 6H), 3.18 (s 3H), 3.75 (d J 8 2H), 4.60 (s 2H), 6.32 (s 2H), 7.71 (s 1H), 7.82 (s 1H), 8.50 (s 1H), 9.42 (s 1H), 13.50 (s 1H).

Example 49: δ 0.12-0.25 (m 4H), 1.02-1.10 (m 1H), 3.20 (s 3H), 3.68 (d J 7 2H), 4.00 (s 3H), 4.80 (s 2H), 7.70 (d J 9 1H), 8.21 (d J 9 1H), 8.38 (s 1H), 9.20 (s 1H), 13.10 (s 1H).

### EXAMPLE 86

### 3-Isobutyl-1-methyl-8-[1-(6-methyl-5-oxo-5,6-dihydro-[1,3]dioxolo[4,5-.g.]isoquinolin-8-yl)-ethyl]-3,7-dihydro-purine-2,6-dione

Benzo[1,3]dioxol-5-ylmethyl-(2,2-dimethoxy-ethyl)-amine (Tetrahedron, 1968, 24:1467) is treated with pyruvic acid according to the general procedure for Intermediate 22 to afford 2-[1,3]dioxolo[4,5-.g.]isoquinolin-8-yl-propionic acid hydrochloride, mp 224-226° C. Treatment with HCl gas in ethanol affords the corresponding ethyl ester hydrochloride, mp 223-225° C. A solution of this compound (2.73 g, 10 mmol) in benzene (20 mL) is treated with dimethyl sulfate (1.26 g, 10 mmol), stirred at room temperature for 5 hours and the solvent is evaporated. The crude oil is dissolved in water (20 mL), cooled to 0-5° C. and a solution of K₃Fe(CN)₆ (5.72 g, 17.4 mmol) in water (25 mL) and sodium hydroxide (2.04 g, 51 mmol) in water (15 mL) are added. After 1.5 hours at 5° C., the reaction is adjusted to pH 2 with concentrated hydrochloric acid and the product collected by filtration then crystallised from methanol-dichloromethane to afford 2-(6-methyl-5-oxo-5,6-dihydro-[1,3]dioxolo[4,5-.g.]isoquinolin-8-yl)-propionic acid, mp 290° C. (dec). This intermediate is then converted to the xanthine according to the general procedure of Method D, [MH]⁺ 452.

### EXAMPLE 87

### 8-(6,7-Dimethoxy-quinolin-4-ylmethyl)-3-isobutyl-1-methyl-3,7-dihydro-purine-2,6-dione

Lithium diisopropylamide (2 M pentane solution, 2.46 mL, 4.92 mmol) and potassium t-butoxide (0.552 g, 4.92 mmol) are added to THF (10 mL) at −70° C., followed by 6,7-dimethoxy-4-methyl-quinoline (J. Org. Chem., 1997, 623:568) (1.0 g, 4.92 mmol). After 1 hour, the reaction is poured on to an excess of crushed dry ice and warmed to room temperature overnight. Pyridine hydrochloride (0.57 g, 4.92 mmol) is added and the reaction partitioned between ether and water. The aqueous phase is evaporated, taken into hot methanol, treated with charcoal, filtered through celite and evaporated to afford (6,7-dimethoxy-quinolin-4-yl)-acetic acid, [MH]⁺ 248. This intermediate is then converted to the xanthine acording to the general procedure of Method C, mp>250° C.

Another aspect of the present invention relates to the treatment of sexual dysfunction, especially male erectile dysfunction (MED) and a cardiovascular disease or disorder comprising administration of a therapeutically effective amount of a pharmaceutical composition comprising a PDE5 inhibitor and an anti-hypertensive agent to a warm-blooded mammal in need thereof. To evaluate the antihypertensive activity of the combination according to the invention, for example, the methodology as described by Lovenberg W, “Animal Models for Hypertension Research”, Prog. Clin. Biol. Res., 1987, 229:225-240 may be applied. For the evaluation that the combination according to the present invention may be used for the treatment of congestive heart failure, for example, the methods as disclosed by Smith H J, Nuttall A, “Experimental Models of Heart Failure”, Cardiovasc. Res., 1985, 19:181-186 may be applied. Molecular approaches such as transgenic methods are also described, for example, by Luft et al., “Hypertension-Induced End-Organ Damage. A New Transgemic Approach For an Old Problem”, Hypertension, 1999, 33: 212-218.

Another aspect of the present invention relates to the treatment of MED and a diabetic disease or disorder comprising administration of a therapeutically effective amount of a pharmaceutical composition comprising a PDE5 inhibitor and an anti-diabetic agent to a warm-blooded mammal in need thereof. The insulin secretion enhancing properties of the combination according to the present invention may be determined by following the methodology as disclosed, for example, in the publication of Tlkenoue et al., Biol. Pharm. Bull., 1997, 29(4):354-359.

Another aspect of the present invention relates to the treatment of MED and a hyperlipidemic disease or disorder comprising administration of a therapeutically effective amount of a pharmaceutical composition comprising a PDE5 inhibitor and an HMG-CoA reductase inhibitor to a warm-blooded mammal in need thereof. To evaluate the HMG-Co-A reductase inhibitory activities of the combination according to the invention, for example, may be determined by following the methodology as disclosed, for example, in U.S. Pat. No. 4,739,073 or U.S. Pat. No. 5,354,772, respectively. The corresponding subject matter of these two references is herewith incorporated by reference in this specification.

In yet another aspect of the present invention relates to the treatment of MED and a metabolic disease or disorder comprising administration of a therapeutically effective amount of a pharmaceutical composition comprising a PDE5 inhibitor and an SSRI to a warm-blooded mammal in need thereof.

The pharmaceutical activities as effected by administration of the combination of active agents used according to the present invention can be demonstrated, e.g., by using corresponding pharmacological models known in the pertinent art. The person skilled in the pertinent art is fully enabled to select a relevant animal test model to prove the hereinbefore and hereinafter indicated therapeutic indications and beneficial effects.

Accordingly, the combination according to the present invention may be used, e.g., for the prevention, delay of progression or treatment of diseases and disorders selected from the group consisting of hyperglycemia, hyperinsulinaemia, hyperlipidaemia, hypertriglyceridemia, diabetes, insulin resistance, impaired glucose metabolism, conditions of impaired glucose tolerance (IGT), conditions of impaired fasting plasma glucose, obesity, diabetic retinopathy, diabetic nephropathy, glomerulosclerosis, diabetic neuropathy, syndrome X, erectile dysfunction, coronary heart disease, hypertension, especially ISH, angina pectoris, myocardial infarction, stroke, vascular restenosis, endothelial dysfunction, impaired vascular compliance, congestive heart failure.

A “diabetic disease or disorder” as defined in this application comprises, but is not limited to, hyperglycemia, hyperinsulinaemia, diabetes, insulin resistance, impaired glucose metabolism, conditions of IGT, conditions of impaired fasting plasma glucose, obesity, diabetic retinopathy, diabetic nephropathy, glomerulosclerosis, diabetic neuropathy and syndrome X.

A “hyperlipidemic disease or disorder” as defined in this application comprises, but is not limited to, hyperlipidaemia, hypertriglyceridemia, coronary heart disease, vascular restenosis, endothelial dysfunction, obesity and impaired vascular compliance.

A “metabolic disease or disorder” as defined in this application comprises, but is not limited to, obesity.

A “cardiovascular disease or disorder” as defined in this application comprises, but is not limited to, hypertension, congestive heart failure, diabetes, glomerulosclerosis, chronic renal failure, coronary heart disease, angina pectoris, myocardial infarction, stroke, vascular restenosis endothelial dysfunction, impaired vascular compliance and congestive heart failure.

Hypertension, especially in connection with a “cardiovascular disease or condition” includes, and is not limited to, mild, moderate and severe hypertension as defined in Journal of Hypertension, 1999, 17:151-183, especially on page 162. Especially preferred is “isolated systolic hypertension” (ISH).

Preferably, the jointly therapeutically effective amounts of the active agents according to the combination of the present invention can be administered simultaneously or sequentially in any order, e.g., separately or in a fixed combination.

All the more surprising is the experimental finding that the combined administration of a PDE5 inhibitor with an anti-diabetic agent, a HMG-Co A reductase inhibitor, an anti-hypertensive agent and/or an SSRI, in each case, a pharmaceutically acceptable form thereof, results not only in a beneficial, especially a potentiating or a synergistic, therapeutic effect. Independent thereof, additional benefits resulting from combined treatment can be achieved such as a surprising prolongation of efficacy, a broader variety of therapeutic treatment and surprising beneficial effects on diseases and conditions.

The term “potentiation” shall mean an increase of a corresponding pharmacological activity or therapeutical effect, respectively. Potentiation of one component of the combination according to the present invention by co-administration of an other component according to the present invention means that an effect is being achieved that is greater than that achieved with one component alone or that is greater than the sum of effects of each component.

The term “synergistic” shall mean that the drugs, when taken together, produce a total joint effect that is greater than the sum of the effects of each drug when taken alone.

Further benefits are that lower doses of the individual drugs to be combined according to the present invention can be used to reduce the dosage, for example, that the dosages need not only often be smaller but are also applied less frequently, or can be used in order to diminish the incidence of side effects. This is in accordance with the desires and requirements of the patients to be treated.

The present invention also relates to a method for the prevention, delay of progression or treatment of sexual dysfunction, especially MED, and a diabetic, cardiovascular, metabolic, hyperlipidemic disease and disorder comprising administering to a warm-blooded mammal, including man, in need thereof jointly therapeutically effective amounts of a pharmaceutical composition comprising

(a) a PDE 5 inhibitor or a pharmaceutically acceptable salt thereof; and

(b) at least one active ingredient selected from the group consisting of

(i) an anti-diabetic agent;

(ii) HMG-Co-A reductase inhibitors;

(iii) an anti-hypertensive agent; and

(iv) a serotonin reuptake inhibitor (SSRI)

or, in each case, a pharmaceutically acceptable salt thereof; and

a pharmaceutically acceptable carrier.

The pharmaceutical composition according to the present invention as described hereinbefore and hereinafter may be used for simultaneous use or sequential use in any order, e.g., for separate use or as a fixed combination.

The pharmaceutical composition according to the present invention comprises a “kit of parts” in the sense that the components can be dosed independently or by use of different fixed combinations with distinguished amounts of the components at different time points. The parts of the “kit of parts” can then, e.g., be administered simultaneously or chronologically staggered, that is at different time points and with equal or different time intervals for any part of the “kit of parts”. Preferably, the time intervals are chosen such that the effect on the treated disease or condition in the combined use of the parts is larger than the effect that would be obtained by use of only any one of the components. Preferably, there is at least one beneficial effect, e.g., a mutual enhancing of the effect of a pharmaceutical composition comprising

(a) a PDE 5 inhibitor or a pharmaceutically acceptable salt thereof; and

(b) at least one active ingredient selected from the group consisting of

(i) an anti-diabetic agent;

(ii) HMG-Co-A reductase inhibitors;

(iii) an anti-hypertensive agent; and

(iv) a serotonin reuptake inhibitor (SSRI)

or, in each case, a pharmaceutically acceptable salt thereof;

and a pharmaceutically acceptable carrier;

in particular a potentiation or a synergism, e.g., a more than additive effect, additional advantageous effects, less side effects, a combined therapeutical effect in a non-effective dosage of one or each of the components, especially a potentiation or synergism.

The invention furthermore relates to a commercial package comprising the combination according to the present invention together with instructions for simultaneous, separate or sequential use.

These pharmaceutical preparations are for oral administration to homeotherms, with the preparations comprising the pharmacological active compound either alone or together with customary pharmaceutical auxiliary substances. For example, the pharmaceutical preparations consist of from about 0.1%-90%, preferably of from about 1% to about 80%, of the active compound. These are prepared in a manner that is known per se, for example using conventional mixing, granulation, coating, solubulizing or lyophilizing processes. Thus, pharmaceutical preparations for oral use can be obtained by combining the active compound with solid excipients, if desired granulating a mixture which has been obtained, and, if required or necessary, processing the mixture or granulate into tablets or coated tablet cores after having added suitable auxiliary substances.

The dosage of the active compound can depend on a variety of factors, such as mode of administration, homeothermic species, age and/or individual condition.

Preferred dosages for the active ingredients of the pharmaceutical combination according to the present invention are therapeutically effective dosages, especially those that are commerically available.

Normally, in the case of oral administration of pharmaceutical composition in accordance with the present invention, an approximate daily dose of from about 1 mg to about 360 mg is to be estimated, preferably a daily dose of from 1-100 mg, more preferably a daily dose of from 1-50 mg, e.g., for a patient of approximately 75 kg in weight.

The insulin secretion enhancer repaglinde is preferably administered in a dosage range of about 0.01 mg to about 8 mg, more preferred from about 0.5 mg to about 6 mg.

In case of HMG-Co-A reductase inhibitors, preferred dosage unit forms of HMG-Co-A reductase inhibitors are, for example, tablets or capsules comprising, e.g., from about 5 mg to about 120 mg, preferably, when using fluvastatin, for example, 20, 40 or 80 mg (equivalent to the free acid) of fluvastatin, for example, administered once a day.

In case of ACE inhibitors, preferred dosage unit forms of ACE inhibitors are, for example, tablets or capsules comprising, e.g., from about 5 mg to about 20 mg, preferably 5, 10, 20 or 40 mg, of benazepril; from about 6.5-100 mg, preferably 6.25, 12.5, 25, 50, 75 or 100 mg, of captopril; from about 2.5 mg to about 20 mg, preferably 2.5, 5, 10 or 20 mg, of enalapril; from about 10 mg to about 20 mg, preferably 10 or 20 mg, of fosinopril; from about 2.5 mg to about 4 mg, preferably 2 or 4 mg, of perindopril; from about 5 mg to about 20 mg, preferably 5, 10 or 20 mg, of quinapril; or from about 1.25 mg to about 5 mg, preferably 1.25, 2.5 or 5 mg, of ramipril. Preferred is t.i.d. administration.

In case of SSRIs, preferred dosage unit forms are, for example, tablets or capsules comprising, e.g., from about 20 mg to about 200 mg, administered once a day.

In case of PDE5, preferred dosage unit forms are, for example, tablets or capsules comprising, e.g., from about 25 mg to about 200 mg, per dose, with 3-isobutyl-8-(6-methoxy-isoquinolin-4-ylmethyl)-1-methyl-3,7-dihydro-purine-2,6-dione being administered in a dose of about 100 mg to about 200 mg.

What is claimed is:

1. A pharmaceutical composition, comprising
(a) a PDE 5 inhibitor or a pharmaceutically acceptable salt thereof and
(b) at least one active ingredient selected from the group consisting of
(i) an anti-diabetic agent;
(ii) HMG-Co-A reductase inhibitors;
(iii) an anti-hypertensive agent; and
(iv) a serotonin reuptake inhibitor (SSRI)
or, in each case, a pharmaceutically acceptable salt thereof; and
a pharmaceutically acceptable carrier.

2. The pharmaceutical composition according to claim 1 wherein PDE 5 inhibitor is a compound of formula 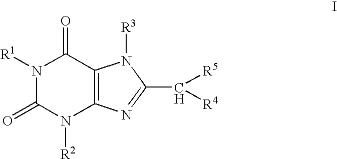
in free or salt form, where
R¹ is hydrogen or alkyl optionally substituted by hydroxy, alkoxy, or alkylthio;
R² is hydrogen, alkyl, hydroxyalkyl, alkylcarbonyloxyalkyl, alkoxyalkyl, alkylthioalkyl, alkenyl, cycloalkylalkyl, heterocyclylalkyl, aralkyl in which the aryl ring thereof is optionally fused to a 5-membered heterocyclic group or is optionally substituted by one or more substituents selected from alkoxy, amino, alkylamino, dialkylamino, acylamino, halogen, hydroxy, aminosulfonyl, alkylaminosulfonyl, dialkylaminosulfonyl, alkylsulfonylamino or dialkylaminosulfonylamino;
R³ is hydrogen or alkyl optionally substituted by hydroxy, alkoxy, or alkylthio;
R⁴ is hydrogen or alkyl;
R⁵ is a quinolinyl, isoquinolinyl or oxodihydroisoquinolinyl group optionally fused to a 5-membered heterocyclic group and optionally substituted by one or more substituents selected from halogen, cyano, hydroxy, alkyl, hydroxyalkyl, alkoxyalkyl, alkylthioalkyl, alkoxy, alkylthio, alkenyl, alkoxycarbonyl, alkynyl, carboxyl, acyl, a group of formula —N(R⁶)R⁷, aryl optionally substituted by one or more substituents selected from halogen or alkoxy, or heteroaryl having 5 or 6 ring atoms attached through a ring carbon atom to the indicated carbon atom; and
R⁶ and R⁷ are each independently hydrogen or alkyl optionally substituted by hydroxy or alkoxy or one of R⁶ and R⁷ is hydrogen and the other is acyl, or R⁶ and R⁷ together with the nitrogen atom to which they are attached denote a 5- or 6-membered heterocyclyl group.

3. The pharmaceutical composition of claim 2, wherein the PDE 5 inhibitor is 3-isobutyl-8-(6-methoxy-isoquinolin-4-ylmethyl)-1-methyl-3,7-dihydro-purine-2,6-dione.

4. The pharmaceutical composition of claim 1, wherein the anti-diabetic agent is selected from the group consisting of insulin secretion enhancers, insulin sensitivity enhancers, insulin signalling pathway modulators, like inhibitors of protein tyrosine phosphatases (PTPases), antidiabetic non-small molecule mimetic compounds and inhibitors of glutamine-fructose-6-phosphate amidotransferase (GFAT); compounds influencing a dysregulated hepatic glucose production, like inhibitors of glucose-6-phosphatase (G6Pase), inhibitors of fructose-1,6-bisphosphatase (F-1,6-BPase), inhibitors of glycogen phosphorylase (GP), glucagon receptor antagonists and inhibitors of phosphoenolpyruvate carboxykinase (PEPCK); pyruvate dehydrogenase kinase (PDHK) inhibitors; inhibitors of gastric emptying; insulin; inhibitors of GSK-3; retinoid X receptor (RXR) agonists; agonists of Beta-3 AR; agonists of uncoupling proteins (UCPs); non-glitazone type PPARγ agonists; dual PPARγ/PPARα agonists; antidiabetic vanadium containing compounds; incretin hormones, like glucagon-like peptide-1 (GLP-1) and GLP-1 agonists; β-cell imidazoline receptor antagonists; miglitol; and α₂-adrenergic antagonists.

5. The pharmaceutical composition of claim 1, wherein the HMG-Co-A reductase inhibitor is selected from the group consisting of atorvastatin, cerivastatin, fluvastatin, pitavastatin, lovastatin, pravastatin, rosuvastatin and simvastatin.

6. The pharmaceutical composition of claim 1, wherein the anti-hypertensive agent is selected from the group consisting of ACE inhibitors, AT1 receptor antagonists, adrenergic blockers, diuretics, neutral endo-peptidases inhibitors, endothelin converting enzymes inhibitors, endothelin receptor antagonists, adrenergic stimulants, alpha/beta adrenergic blockers beta adrenergic blocking agents, calcium channel blockers, diuretics, rauwolfia derivatives and vasodilators.

7. The pharmaceutical composition of claim 1, wherein the SSRI is selected from the group consisting of fluvoxamine, fluoxetine, paroxetine, sertraline, citalopram, venlafaxine, cericlamine, duloxetine, milnacipran, nefazodone and cyanodothiepin.

8. A method for the prevention, delay of progression or treatment of sexual dysfunction, hyperglycemia, hyperinsulinaemia, hyperlipidaemia, hypertriglyceridemia, diabetes, insulin resistance, impaired glucose metabolism, conditions of impaired glucose tolerance (IGT), conditions of impaired fasting plasma glucose, obesity, diabetic retinopathy, diabetic nephropathy, glomerulosclerosis, diabetic neuropathy, syndrome X, erectile dysfunction, coronary heart disease, hypertension, especially ISH, angina pectoris, myocardial infarction, stroke, vascular restenosis, endothelial dysfunction, impaired vascular compliance, congestive heart failure, comprising administering a therapeutically effective amount of the pharmaceutical composition of claim 1 to a warm-blooded mammal in need thereof.

9. The method of claim 8, wherein sexual dysfunction is male erectile dysfunction.
